SEQUENCE LISTING (20 sequences)

SEQ ID NO: 1 - protein, 10 aa, Homo sapiens
ELPSFLGKRT

SEQ ID NO: 2 - protein, 11 aa, Homo sapiens
EGFPDKQPRKK

SEQ ID NO: 3 - protein, 16 aa, synthetic construct
  REGION 1..16: note = CDRL1 region of the 5C3 monoclonal antibody
RSSQSIVHSNGNTYLE

SEQ ID NO: 4 - protein, 7 aa, synthetic construct
  REGION 1..7: note = CDRL2 region of the 5C3 monoclonal antibody
KVSNRLS

SEQ ID NO: 5 - protein, 9 aa, synthetic construct
  REGION 1..9: note = CDRL3 region of the 5C3 monoclonal antibody
FQGSHVPFT

SEQ ID NO: 6 - protein, 5 aa, synthetic construct
  REGION 1..5: note = CDRH1 region of the 5C3 monoclonal antibody
ETYMH

SEQ ID NO: 7 - protein, 17 aa, synthetic construct
  REGION 1..17: note = CDRH2 region of the 5C3 monoclonal antibody
RIDPANGNTKDDPKFQG

SEQ ID NO: 8 - protein, 6 aa, synthetic construct
  REGION 1..6: note = CDRH3 region of the 5C3 monoclonal antibody
SYAMDY

SEQ ID NO: 9 - protein, 23 aa, synthetic construct
  REGION 1..23: note = FRL1 region of the 5C3 monoclonal antibody
DVLMTQTPLSLPVSLGDQASISC

SEQ ID NO: 10 - protein, 15 aa, synthetic construct
  REGION 1..15: note = FRL2 region of the 5C3 monoclonal antibody
WYLQKTGQSPELLIY

SEQ ID NO: 11 - protein, 32 aa, synthetic construct
  REGION 1..32: note = FRL3 region of the 5C3 monoclonal antibody
GVPDRFSGSGSGTDFTLKISRVEAEDLGVYYC

SEQ ID NO: 12 - protein, 10 aa, synthetic construct
  REGION 1..10: note = FRL4 region of the 5C3 monoclonal antibody
FGSGTKLEIK

SEQ ID NO: 13 - protein, 30 aa, synthetic construct
  REGION 1..30: note = FRH1 region of the 5C3 monoclonal antibody
EAQLQQSGAELVKPGASVKLSCTASGFNIQ

SEQ ID NO: 14 - protein, 14 aa, synthetic construct
  REGION 1..14: note = FRH2 region of the 5C3 monoclonal antibody
WVKQRPEQGLEWIG

SEQ ID NO: 15 - protein, 32 aa, synthetic construct
  REGION 1..32: note = FRH3 region of the 5C3 monoclonal antibody
KASITVDTSSNTAYLQLSSLTSEDTAVYYCAS

SEQ ID NO: 16 - protein, 11 aa, synthetic construct
  REGION 1..11: note = FRH4 region of the 5C3 monoclonal antibody
WGQGTSVTVSS

SEQ ID NO: 17 - protein, 112 aa, synthetic construct
  REGION 1..112: note = VL region of the 5C3 monoclonal antibody
DVLMTQTPLSLPVSLGDQASISCRSSQSIVHSNGNTYLEWYLQKTGQSPELLIYKVSNRLSGVPDRFSGSGSGTDFTLKISRVEAEDLGVYYCFQGSHVPFTFGSGTKLEIK

SEQ ID NO: 18 - protein, 115 aa, synthetic construct
  REGION 1..115: note = VH region of the 5C3 monoclonal antibody
EAQLQQSGAELVKPGASVKLSCTASGFNIQETYMHWVKQRPEQGLEWIGRIDPANGNTKDDPKFQGKASITVDTSSNTAYLQLSSLTSEDTAVYYCASSYAMDYWGQGTSVTVSS

SEQ ID NO: 19 - protein, 101 aa, Homo sapiens
MACPLEKALDVMVSTFHKYSGKEGDKFKLNKSELKELLTRELPSFLGKRTDEAAFQKLMSNLDSNRDNEVDFQEYCVFLSCIAMMCNEFFEGFPDKQPRKK

SEQ ID NO: 20 - protein, 104 aa, synthetic construct
  REGION 1..104: note = Recombinant S100A4 including an N-terminal end
GSHMACPLEKALDVMVSTFHKYSGKEGDKFKLNKSELKELLTRELPSFLGKRTDEAAFQKLMSNLDSNRDNEVDFQEYCVFLSCIAMMCNEFFEGFPDKQPRKK

(19) United States
(12) Patent Application Publication
(10) Pub. No.: US 2025/0011405 A1
(43) Pub. Date: Jan. 9, 2025

(54) TREATMENT AND DIAGNOSIS OF DISEASES ASSOCIATED TO PATHOGENIC FIBROSIS

(71) Applicant: LYKERA BIOMED, S.A., Barcelona (ES)

(72) Inventors: Francesc Mitjans Prat, Barcelona (ES); José Luis HERNÁNDEZ MÍGUEZ, Barcelona (ES); Jaume ADAN PLANA, Barcelona (ES); Ramon MESSEGUER PEYPOCH, Barcelona (ES); Marc MASA ÁLVAREZ, Barcelona (ES); Josep Maria MARTÍNEZ ESCOLÀ, Barcelona (ES)

(21) Appl. No.: 18/711,513

(22) PCT Filed: Nov. 18, 2022
(86) PCT No.: PCT/EP2022/082483

§ 371 Date: May 17, 2024

(30) Foreign Application Priority Data
Nov. 19, 2021 (EP) 21383047.4

(51) Int. Cl. C07K 16/18; A61K 39/00; A61P 9/00; G01N 33/53; G01N 33/68

(52) U.S. Cl. CPC C07K 16/18; A61P 9/00; G01N 33/53; G01N 33/6893; A61K 2039/505; C07K 2317/34; C07K 2317/565; C07K 2317/567; G01N 2800/32; G01N 2800/7052

(57) ABSTRACT

The invention relates to an antibody or a fragment thereof that binds specifically to the S100A4 protein for use in the prevention and/or treatment of a disease associated to pathogenic fibrosis. The invention also relates to in vitro methods for diagnosing a disease associated to pathogenic fibrosis in a subject or to in vitro methods for designing a customized therapy based on the detection of the levels of the S100A4 protein, and to the use of kits for diagnosing said diseases.

### FIELD OF THE INVENTION

The invention relates to the treatment and diagnosis of diseases associated to pathogenic fibrosis.

### BACKGROUND OF THE INVENTION

Fibrosis is not a static process; extracellular matrix is constantly being laid down and resorbed and the progressive accumulation of fibrous tissue is thought to represent a relative imbalance between pro-fibrotic processes and anti-fibrotic processes. If these processes are not properly regulated, the pathologic and progressive accumulation of collagen and other matrix proteins in the extracellular space as a result of a disordered wound healing process leads to replacement of normal cells by dense fibrous bands of protein, and results in fibrotic disease with disordered function in the affected organ for example, impairment of respiratory function, impaired circulatory function via fibrotic changes in arterial walls, fibrotic degeneration of renal and liver function, degenerative musculoskeletal function, fibrotic degeneration of cardiac muscle or skeletal muscle, fibrotic degenerative changes in neuronal tissues in the central nervous system as well as the peripheral nervous system.

Several therapies are administered to patients suffering from pathogenic fibrosis such as treatment with steroids, and cytotoxic drugs such as azathioprine or cyclophosphamide are sometimes added to the steroid treatment. However, a large number of studies have shown little or no benefit of these drugs. There are currently no drugs approved for treatment of pathogenic fibrosis.

Hence, there remains an unmet medical need for therapies for treatment of diseases associated to pathogenic fibrosis. In addition, there is a need in the state of the art to provide further non-invasive methods to diagnose diseases associated to pathogenic fibrosis.

### SUMMARY OF THE INVENTION

In a first aspect, the invention relates to an antibody or a fragment thereof that binds specifically to the S100A4 protein for use in the prevention and/or treatment of a disease associated to pathogenic fibrosis.

In a second aspect, the invention relates to an in vitro method for diagnosing a disease associated to pathogenic fibrosis in a subject which comprises:
a) Detecting the levels of the S100A4 protein or of a variant thereof in a sample of said subject, and
b) Comparing said levels with a reference value,
   wherein increased levels of the S100A4 protein or of a variant thereof with respect to the reference value are indicative of the subject suffering from a disease associated to pathogenic fibrosis.

In a third aspect, the invention relates to an in vitro method for designing a customized therapy for a subject diagnosed with a disease associated to pathogenic fibrosis which comprises:
a) detecting the levels of the S100A4 protein or of a variant thereof in a sample of said subject, and
b) comparing said levels with a reference value,
   wherein increased levels of the S100A4 protein or of a variant thereof with respect to the reference value are indicative that the subject is to be treated with an antibody or a fragment thereof that binds specifically to the S100A4 protein.

In a fourth aspect, the invention relates to the use of a kit for diagnosing a disease associated to pathogenic fibrosis wherein the kit comprises an antibody or a fragment thereof that binds specifically to the S100A4 protein, and a set of reagents suitable for detecting the levels of S100A4.

### DESCRIPTION OF THE FIGURES

FIG. 1: Levels of S100A4 protein in serum or plasma samples. To measure the presence of S100A4 in mice serum or plasma, a sandwich ELISA was performed using antibodies against S100A4 (mAb). Ninety-six microtiter plates were coated with 10 μg/ml of 5C3 mAb. After blocking using PBS containing 1% of non-fat dry milk, conveniently diluted plasma samples were added to the wells and incubated for 2 h at 37° C. Then, rabbit anti-mouse S100A4 polyclonal antibody (DAKO) at 10 μg/ml was added to the wells and incubated for 2 h at 37° C. Finally, streptavidin-horseradish peroxidase was added and incubated for 1 h at 37° C. Signal was developed by adding tetramethylbenzidine substrate and, after stopping the reaction, absorbance was measured at 450 nm using a spectrophotometer. A standard curve was constructed by plotting absorbance values versus rhS100A4 protein concentrations.

FIG. 2: S100A4 increases tissue fibrosis in the liver. Representative images of the picrosirius red-stained liver sections visualized under polarized light. IHC: immunohistochemistry.

FIG. 3: S100A4 protein induces liver fibrosis and 5C3 mAb neutralizes this effect. Images of representative livers of the vehicle PBS-treated group (control), the S100A4-treated group and the group treated with angiotensin followed by mAb 5C3. IHC: immunohistochemistry.

FIG. 4: Angiotensin II induces ventricle hypertrophy and cardiac fibrosis. The images on the top, show the increase in the size of the ventricle after treatment with angiotensin II. The graph on the bottom, shows the correlation between the weight of the left ventricle (LV) and the size of the animals (length of the tibia).

FIG. 5: Biomarker analysis after Angiotensin II treatment. A) mRNA levels of S100A4 analysed by qPCR. B) mRNA levels of RAGE analysed by qPCR. C) Positive correlation between S100A4 expression levels and presence of Col3a1 in treated animals.

FIG. 6: Effect of 5C3 mAb in cardiac fibrosis. Quantification of Systolic blood pressure and left ventricle (LV) weight on treated animals with angiotensin II, 5C3 mAb and angiotensin followed by 5C3 mAb. Graphs show mean±sd. Mann Whitney U-test *p<0.001, ns p>0.05.

FIG. 7: Effect of 5C3 mAb in S100A4 and RAGE expression levels. mRNA expression levels on treated animals with angiotensin II, 5C3 mAb and angiotensin followed by 5C3 mAb analysed by qPCR. Graphs show mean±sd. Mann Whitney U-test *p<0.01, ns p>0.05.

FIG. 8: Effect of 5C3 mAb on the expression levels of different cardiac fibrosis biomarkers (BNP, SktACT, βMHC, Col1a1, Col3a1, Periostin). mRNA expression levels on treated animals with angiotensin II, 5C3 mAb and angiotensin followed by 5C3 mAb analysed by qPCR. Graphs show mean±sd. Mann Whitney U-test *p<0.01, ns p>0.05.

### DESCRIPTION OF THE INVENTION

The authors of the present invention have discovered that the detection of increased levels of S100A4 can be used in a method of diagnosing diseases associated to pathogenic fibrosis (Examples 1 and 3). Moreover, antibodies against S100A4 protein are useful in the treatment of said diseases (Examples 2 and 4).

### Medical Uses

In a first aspect, the invention relates to an antibody or a fragment thereof that binds specifically to the S100A4 protein for use in the prevention and/or treatment of a disease associated to pathogenic fibrosis.

Alternatively, the invention relates to a method for the prevention and/or treatment of a disease associated to pathogenic fibrosis, which comprises administering an antibody or a fragment thereof that binds specifically to the S100A4 protein to a subject in need thereof.

Alternatively, the invention relates to the use of an antibody or a fragment thereof that binds specifically to the S100A4 protein for the preparation of a medicament for the prevention and/or treatment of a disease associated to pathogenic fibrosis.

“Medicament” is understood as a pharmaceutical composition comprising an antibody or a fragment thereof that binds specifically to the S100A4 protein.

“Prevention”, as used herein, refers to a decrease in the occurrence of fibrosis-associated cells that might be responsible for the production and deposition of matrix fibers as a feature of fibrosis in an animal, and/or the matrix deposition itself in an animal. The prevention may be complete (e.g. the total absence of fibrosis-associated cells and/or the matrix deposition itself in a subject). The prevention may also be partial, such that, for example, when the occurrence of fibrosis-associated cells and/or the matrix deposition itself in a subject is less than that which would have occurred without the present invention. Prevention also refers to reduced susceptibility to a clinical condition. In the context of the present invention prevention is understood as the administration of an antibody or a fragment thereof that binds specifically to the S100A4 protein in an initial or early stage of the disease, or to also prevent its onset.

“Treatment”, as used herein, refers to any type of therapy, which aims at terminating, preventing, ameliorating or reducing the susceptibility to a clinical condition as described herein. In a preferred embodiment, the term treatment relates to prophylactic treatment (i.e., a therapy to reduce the susceptibility of a clinical condition) of a disorder or a condition as defined herein. Thus, “treatment”, “treating”, and their equivalent terms refer to obtaining a desired pharmacological or physiological effect, covering any treatment of a pathological condition or disorder in a mammal, including a human. The effect may be prophylactic in terms of completely or partially preventing a disorder or symptom thereof and/or may be therapeutic in terms of a partial or complete cure for a disorder and/or adverse effect attributable to the disorder. That is, “treatment” includes (1) preventing the disorder from occurring or recurring in a subject, (2) inhibiting the disorder, such as arresting its development, (3) stopping or terminating the disorder or at least symptoms associated therewith, so that the host no longer suffers from the disorder or its symptoms, such as causing regression of the disorder or its symptoms, for example, by restoring or repairing a lost, missing or defective function, or stimulating an inefficient process, or (4) relieving, alleviating, or ameliorating the disorder, or symptoms associated therewith, where ameliorating is used in a broad sense to refer to at least a reduction in the magnitude of a parameter, such as inflammation, or pain.

“Disease associated to pathogenic fibrosis” is a disease where pathogenic fibrosis occurs (i.e., when said process is harmful or indesirable), and it relates to any disease giving rise to fibrosis, particularly excessive fibrosis, whether as a main or a secondary symptom. Fibrosis is characterized by the accumulation and reorganization of the extracellular matrix (ECM). Despite having obvious etiological and clinical distinctions, most chronic fibrotic disorders have in common a sustained production of growth factors, proteolytic enzymes, angiogenic factors, and fibrogenic cytokines, which stimulate the deposition of connective tissue elements, especially collagens and proteoglycans, which progressively remodel and destroy normal tissue architecture. Fibrosis is the formation of excess fibrous connective tissue in an organ or tissue in a reparative or reactive process. This can be a reactive, benign, or pathological state. The deposition of connective tissue in the organ and/or tissue can obliterate the architecture and function of the underlying organ or tissue. Pathological fibrosis is this pathological state of excess deposition of fibrous tissue, as well as the process of connective tissue deposition in healing. Pathological fibrosis is characterized by non-resolving or progressive tissue remodeling, which itself can cause tissue damage and organ failure. In a preferred embodiment, the pathogenic fibrosis is associated to increased levels of S100A4.

“Increased levels”, in relation to the expression of S100A4, as used herein, relates to the value of a parameter that measures the degree of expression of a specific gene or of the corresponding polypeptide. In a particular embodiment, said value can be determined by measuring the mRNA level of the gene of interest or a variant thereof or by measuring the amount of protein encoded by said gene of interest or a variant thereof. Thus, in the context of the present invention, in a particular embodiment, said expression level comprises determining the level of the mRNA encoded from the S100A4 gene or determining the level of the S100A4 protein or a variant thereof. The increased levels of S100A4 can be determined by any method disclosed in relation to the diagnostic method of the invention. In a particular embodiment, an increase in S100A4 levels of at least 1.1-fold, 1.5-fold, 5-fold, 10-fold, 20-fold, 30-fold, 40-fold, 50-fold, 60-fold, 70-fold, 80-fold, 90-fold, 100-fold or even more compared with a reference value is considered as “increased” expression. Methods for detecting increased levels of S100A4, and the meaning of a reference value is disclosed in relation to the diagnostic method of the invention and it is equally applicable to this aspect of the invention.

The terms “S100A4”, “S100A4 protein”, “S100 calcium-binding protein A4”, “calcium protein”, “calvasculin”, “metastasin”, “murine placental homolog”, “MTS1”, “CAPL”, “p9Ka”, “18A2”, “pEL98”, “42A”, “FSP1”, “fibroblast-specific protein-1”, “malignant transformation suppression 1”, “leukemia multidrug resistance associated protein”, “OTTHUMP00000015469”, and “OTTHUMP00000032895”, are used interchangeably, and include as well variants, isoforms, species homologs of human or murine S100A4, and analogs having at least one common epitope with human or murine S100A4. The term includes functionally equivalent variants of S100A4, i.e., any molecule sharing with S100A4 one or more of the functions described in the present invention associated with S100A4, particularly the fibrotic activity (such as the increase of collagen fiber crosslinking or the increase in the expression of one or more fibrosis biomarkers selected from the group consisting of BNP, SktAct, βMHC, Col1a1, Col3a1 and periostin), and having a minimal identity in the amino acid sequence. The term also includes all the physiologically relevant post-translational chemical modification forms, for example, glycosylation, phosphorylation or acetylation, etc., provided that the functionality of the protein is maintained. Said term encompasses the S100A4 of any mammal species, including but not being limited to, domestic and farm animals (cows, horses, pigs, sheep, goats, dogs, cats or rodents), primates and humans. Preferably, the S100A4 is human. The complete cDNA sequence for human S100A4 has the Genbank accession number M80563 (31 Oct. 1994). “Human S100A4” is understood as the protein defined by the sequence of the UniProt database with accession number P26447 (release of 2 Jun. 2021), corresponding to the sequence of the human S100A4 protein (SEQ ID NO: 19). In a preferred embodiment, the S100A4 protein is a protein with SEQ ID NO: 19:

MACPLEKALDVMVSTFHKYSGKEGDKFKLNKSELKELLTRELPSFLGKR
TDEAAFQKLMSNLDSNRDNEVDFQEYCVFLSCIAMMCNEFFEGFPDKQP
RKK

The variants of S100A4 can be both natural and artificial. The expression “natural variant” refers to all those variants of human S100A4 mentioned above which occur naturally in other species, i.e., S100A4 orthologs. Said natural variants include but are not limited to, S100A4 of cows, corresponding to the predicted sequence with accession number DAA31755.1 (version of 21 May 2010); S100A4 of rats, corresponding to the predicted sequence with accession number NP_036750.1 (version of 13 Feb. 2021); S100A4 of mouse, corresponding to the predicted sequence with accession number NP_035441.1 (version of 9 Apr. 2021); S100A4 of dogs, corresponding to the predicted sequence with accession number NP_001003161.1 (version of 26 Jun. 2021) or to the predicted sequence with accession number NP_001349526.2 (version of 25 Jun. 2021); S100A4 of cats, corresponding to the predicted sequence with accession number XP_019678117.1 (version of 12 Dec. 2017) or XP_003999828.1 (version of 12 Dec. 2017). The natural variants of S100A4 can also be derived from said sequences by means of insertion, substitution (such as conservative substitution) or deletion of one or more amino acids and include natural alleles, variants resulting from alternative processing and secreted and truncated forms occurring naturally. The S100A4 can also be an artificial variant produced by recombinant and/or synthetic means.

Additionally, the functionally equivalent variants of S100A4 include polypeptides showing at least 60%, 65%, 70%, 72%, 74%, 76%, 78%, 80%, 90%, 95%, 97%, 99% similarity or identity with the different natural variants of S100A4 mentioned above. The degree of identity between two polypeptides is determined using algorithms implemented in a computer and methods which are widely known by the persons skilled in the art. The identity between two amino acid sequences is preferably determined using the BLASTP algorithm (BLAST Manual, Altschul, S. et al., NCBI NLM NIH Bethesda, Md. 20894, Altschul, S., et al., J., 1990, Mol. Biol. 215:403-410).

In a preferred embodiment, the disease is not a pneumoconiosis, preferably is not an occupational lung fibrosis or a lung fibrosis caused by inhalation of chemical substances.

In another preferred embodiment, the disease is not silicon-induced fibrosis, asbestos-induced fibrosis or cystic fibrosis; preferably is not silicon-induced fibrosis or asbestos-induced fibrosis. In another preferred embodiment, the disease is not associated to metastasis. In another preferred embodiment the disease is not lung fibrosis.

In another preferred embodiment, the disease is selected from the group consisting of liver fibrosis, cardiac fibrosis, kidney fibrosis and lung fibrosis; more preferably is liver fibrosis, cardiac fibrosis and kidney fibrosis.

In another preferred embodiment, the disease is cardiac fibrosis. In another preferred embodiment, the disease is liver fibrosis.

In a preferred embodiment, the lung fibrosis is idiopathic pulmonary fibrosis.

“Cardiac fibrosis”, as used herein, relates to the excess deposition of extracellular matrix in the cardiac muscle, but the term may also refer to an abnormal thickening of the heart valves due to inappropriate proliferation of cardiac fibroblasts.

“Lung fibrosis” or “pulmonary fibrosis”, as used herein, relates to a condition in which the lungs become scarred over time. Symptoms include shortness of breath, a dry cough, feeling tired, weight loss, and nail clubbing

In a preferred embodiment the disease associated to pathogenic fibrosis is not a disease associated to an undesired angiogenesis. A disease associated to an undesired angiogenesis relates to all those diseases where pathogenic angiogenesis occur, i.e., when said process is harmful or undesirable.

“Antibody”, as used herein, relates to a monomeric or multimeric protein which comprises at least one polypeptide having the capacity for binding to a determined antigen and comprising all or part of the light or heavy chain variable region of an immunoglobulin molecule. The term antibody includes any type of known antibody, such as, for example, polyclonal antibodies, monoclonal antibodies, and genetically engineered antibodies, such as chimeric antibodies, humanized antibodies, primatized antibodies, human antibodies, bispecific antibodies, single domain antibodies, VHH antibodies, caninized antibodies, felinized antibodies, canine antibodies, and feline antibodies.

The term “antibody” is used herein in the broadest sense and covers monoclonal antibodies (including full length monoclonal antibodies), polyclonal antibodies, multispecific antibodies (e.g. bispecific antibodies), and antibody fragments so long as they exhibit the desired biological activity.

In a preferred embodiment, the antibody or fragment thereof is a neutralizing antibody or fragment thereof.

“Neutralizing” (or an inhibitory antibody or fragment thereof), as used herein, relates to an antibody or fragment thereof that inhibits the fibrotic activity of S100A4. The inhibition can occur by at least 10%, at least 20%, at least 30%, at least 40%, at least 50%, at least 60%, at least 70%, at least 75%, at least 80%, at least 85%, at least 90%, at least 95%, at least 97%, at least 98%, at least 99%, and at least 99.9% or 100%. The fibrotic activity inhibition may be measured by any means known to one of ordinary skill in the art, for example, as measured the reduction of collagen fiber crosslinking or the reduction in the expression of one or more fibrosis biomarkers selected from the group consisting of BNP, SktAct, βMHC, Col1a1, Col3a1 and periostin.

An intact “antibody” includes heteromultimeric glycoproteins comprising at least two heavy (H) chains and two light (L) chains inter-connected by disulfide bonds. Typically, each light chain is linked to a heavy chain by one covalent disulfide bond while the number of disulfide linkages between the heavy chains of different immunoglobulin isotypes varies. Each heavy and light chain also has intrachain disulfide bridges. Each heavy chain has at one end a heavy chain variable region (abbreviated herein as HCVR or VH) followed by a heavy chain constant region. The heavy chain constant region is comprised of three domains, CH1, CH2, and CH3. Each light chain has a light chain variable region (abbreviated herein as LCVR or VL) and a light chain constant region at its other end. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions are not directly involved in the binding of the antibody to the antigen but exhibit various effector functions such as participation in antibody dependent cell-mediated cytotoxicity (ADCC), phagocytosis via binding to Fcγ receptor, half-life/clearance rate via neonatal Fc receptor (FcRn) and complement dependent cytotoxicity via the C1q component of the complement cascade.

The invention also comprises the use of fragments of the different types of antibodies mentioned above which substantially preserve the anti-fibrotic activity of the antibody.

The term “fragment” when referring to an antibody, means antigen binding fragments of an antibody comprising a partial heavy or light chain variable sequence, which retain capacity to bind human or murine S100A4. In a preferred embodiment, the fragment includes the sequence of the 6 CDRs regions. In another embodiment, the fragment includes the sequence of the 3 CDRs in the case of single chain antibodies like VHH or nanobodies. Examples of fragments include, without being limited to, (i) a Fab fragment, a monovalent fragment consisting of the VL, VH, CL, and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody; (v) a dAb fragment (Ward et al., (1989) Nature 341:544-546), which consists of a VH domain; and (vi) an isolated complementarity determining region (CDR). Fragments can be prepared by recombinant techniques or enzymatic or chemical cleavage of intact antibodies, e.g. papain digestion (see for example, WO94/29348). The term “antibody fragment” includes antibody fragments such as Fab, F(ab′)₂, Fab′, single chain Fv fragments (scFv), diabodies and nanobodies

The Fab and F(ab′)₂ fragments can be obtained by means of enzymatic or chemical cleavage of the intact antibodies. Papain digestion of antibodies produces two identical antigen binding fragments referred to as “Fab” fragments, each with a single antigen binding site, and a residual “Fc” fragment, the name of which reflects its capacity for readily crystallizing. Pepsin treatment yields an F(ab′)₂ fragment which has two antigen binding sites and which is still capable of cross-linking to the antigen.

“Fv” is the minimal antibody fragment containing a complete antigen binding and antigen recognition site. This region consists of a variable domain of a variable light chain and heavy chain dimer in a strong noncovalent association. In this configuration the three hypervariable regions of each variable domain interact to define an antigen binding site on the surface of the VH-VL dimer. As a whole, the six hypervariable regions confer antigen-antibody specificity to the antibody. However, even a single variable domain (or half an Fv, which comprises only three hypervariable regions specific for an antigen) has antigen recognition and binding capacity, although with less affinity than the complete binding site.

The Fab fragment also contains the constant domain of the light chain and the first constant domain (CH1) of the heavy chain. Fab′ fragments differ from Fab fragments in the addition of a few residues at the carboxy terminus of the domain CH1 of the heavy chain, including one or more cysteines of the antibody hinge region.

The “single chain Fv” or “scFv” antibody fragments comprise the VH and VL domains of an antibody, in which these domains are present in a single polypeptide chain. Preferably, the Fv polypeptide additionally comprises a linker polypeptide between the VH and VL domains which allows the scFv to form the desired structure for antigen binding. For a review of scFv, see Pluckthun in The Pharmacology of Monoclonal Antibodies, vol. 113, Rosenburg and Moore eds., Springer-Verlag, N.Y., pp. 269-315 (1994).

The term “diabodies” refers to small antibody fragments with two antigen binding sites, those fragments comprising a heavy chain variable domain (VH) connected to a light chain variable domain (VL) in the same polypeptide chain (VH-VL). By means of using a linker which is too short to allow pairing between the two domains in the same chain, the domains are forced to pair with the complementary domains of another chain and create two antigen binding sites. Diabodies are described in further detail in, for example, documents EP 404,097; WO 93/11161; and Hollinger et al., Proc. Natl. Acad. Sci. USA, 90: 6444-6448 (1993).

The term “nanobodies” designates small sized entities (15 kDa) formed solely by the antigen binding region of the heavy chain (VH fragment) of immunoglobulins. Said nanobodies are mainly produced after immunizing animals of the Camelidae family, such as camels, llamas and dromedaries, mainly llamas; and also of the shark family, which have the particularity of having antibodies which naturally lack the light chain and recognize the antigen by the heavy chain variable domain. Nevertheless, the nanobodies derived from these sources require a humanization process for their therapeutic application. Another potential source for obtaining nanobodies is from antibodies derived from different human samples by separating the VH and VL domains of the variable region. Nanobodies present advantages such as a production cost reduction with respect to whole antibodies, stability and the reduction of immunogenicity.

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); See, e.g., Bird et al., (1988) Science 242:423-426; and Huston et al. (1988) Proc. Natl. Acad. Sci. USA 85:5879-5883). Such single chain antibodies are included by reference to the term “antibody”.

In a preferred embodiment, the antibody is a monoclonal antibody. The term “monoclonal antibody” refers to a preparation of antibody molecules of homogeneous molecular composition. i.e., the individual antibodies comprising the population are identical except for possible naturally occurring mutations that may be present in minor amounts. A monoclonal antibody composition displays a single binding specificity and affinity for a particular epitope or antigenic binding site. The monoclonal antibodies are produced by a hybrid cell product of the fusion of a B-cell clone descendent of a single unique parent cell and a tumor plasma cell. Furthermore, in contrast to polyclonal antibody preparations that typically include different antibodies directed against different determinants (epitopes), each monoclonal antibody is directed against a single determinant on the antigen.

The term “diclonal antibody” refers to a preparation of at least two antibodies to S100A4, preferably to murine, canine, feline, or human S100A4. Typically, the different antibodies bind different epitopes.

The term “oligoclonal antibody” refers to a preparation of 3 to 100 different antibodies to S100A4, preferably to murine, canine, feline, or human S100A4. Typically, the antibodies in such a preparation bind to a range of different epitopes.

The term “polyclonal antibody” refers to a preparation of more than 1 (two or more) different antibodies to S100A4, preferably to murine, canine, feline, or human S100A4 derived from different B-cell lines, i.e., antibodies which are a mixture of immunoglobulins, secreted against a specific antigen (S100A4). Such a preparation includes antibodies binding to a range of different epitopes.

The term “bispecific antibody” refers to that antibody having two different binding specificities, see. e.g., U.S. Pat. Nos. 5,922,845 and 5,837,243; Zeilder (1999) J. Immunol. 163:1246-1252; Somasundaram (1999) Hum. Antibodies 9:47-54; Keler (1997) Cancer Res. 57:4008-4014. For example, a bispecific antibody may have one binding site for a cell surface antigen, and a second binding site for an Fc receptor on the surface of an effector cell. The exemplary bispecific antibodies can bind to two different epitopes of the B-cell surface marker. Others of the said antibodies can bind to a first B-cell marker and additionally bind to a second B-cell surface marker. Alternatively, a binding arm of an anti-B cell marker can be combined with an arm which binds to a triggering molecule in a leukocyte, such as a T-cell receptor molecule (for example, CD2 or CD3), or Fc receptors for IgG (FcγR), such as FcγRI (CD64), FcγRII (CD32) and FcγRIII (CD 16), such that the mechanisms of cell defense are concentrated in the B-cell. Bispecific antibodies can also be used to locate cytotoxic agents against the B-cell. These antibodies have a binding arm to the marker of the lymphocyte and an arm which binds to the cytotoxic agent (for example, saporin, anti-interferon-α, vinca alkaloid, ricin A-chain, methotrexate or a radioactive hapten isotope). Bispecific antibodies can be prepared as whole antibodies or as antibody fragments (for example, F(ab)2 bispecific antibodies).

Bispecific antibodies further include diabodies. Diabodies are bivalent, bispecific antibodies in which the VH and VL domains are expressed on a single polypeptide chain, but using a linker that is too short to allow for pairing between the two domains on the same chain, thereby forcing the domains to pair with complementary domains of another chain and creating two antigen binding sites (See, e.g., Holliger, P., et al., (1993) Proc. Natl. Acad. Sci. USA 90:6444-6448; Poljak, R J., et al., (1994) Structure 2:1121-1123).

The term “multispecific antibody” refers to that antibody having at least three binding sites or specificities.

The term “epitope” refers to an antigenic determinant capable of specific binding to an antibody, or the place where an antibody binds its antigen, or by extension to the peptide presented in an MHC molecule to which a T-cell receptor binds. One and the same antigen can have different epitopes. Epitopes consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents.

The term “isolated” means identified and separated or removed from its natural environment. For example, a polynucleotide or a polypeptide naturally present in a living organism is not “isolated,” but the same polynucleotide or polypeptide separated from the coexisting materials of its natural state is “isolated,” including but not limited to when such polynucleotide or polypeptide is introduced back into a cell, even if the cell is of the same species or type as that from which the polynucleotide or polypeptide was separated.

“Chimeric antibodies” are understood as antibodies constructed with variable regions of an antibody of a species (usually a mammal in which the monoclonal antibody was generated) and constant regions of another species (that species in which the chimeric antibody is going to be used). The objective of said construct is to obtain an antibody with the original monoclonal antibody but which is less immunogenic and better tolerated in the subject who is going to be treated, with an improved serum half-life and which can be recognized by immunological effector mechanisms, i.e., the complement, the Fc receptor of cytotoxic cells or other specific immunoglobulin receptors which show species specificity. In a preferred embodiment, the chimeric antibodies are formed by murine variable regions and human constant regions.

“Humanized antibody” is understood as an antibody from a non-human organism, typically a murine antibody, which conserves the antigen binding properties of the parent antibody, but which is less immunogenic in human beings. This can be achieved by means of different processes, which include (a) grafting the complete nonhuman variable domains into human constant regions to generate chimeric antibodies; (b) grating only the nonhuman complementarity determining regions (CDR) in a human framework and the constant regions, with or without retaining the critical framework residues; and (c) transplanting the complete nonhuman variable domains, but “concealing them” with a section similar to the human variable domain by means of replacing the surface residues.

“Primatized antibody” is understood as a recombinant antibody that has been genetically manipulated to contain the heavy and light variable domains of a monkey antibody (or of another primate), particularly an antibody of a cynomolgus monkey, and containing sequences of a human constant domain, preferably the constant domain of human gamma 1 or 4 immunoglobulin (or a PE variant). The preparation of said antibodies is described in Newman et al., Biotechnology, 10: 1458-1460 (1992); and in patent documents U.S. Pat. Nos. 5,658,570 and 6,113,898. It has been described that these antibodies show a high degree of homology or identity with human antibodies, i.e., 85-98%, they have human effector functions, they have lower immunogenicity and can show a high affinity for human antigens. Another very effective means for generating recombinant antibodies is described by Newman, Biotechnology, 10: 1455-1460 (1992).

“Human antibody” is understood as an antibody containing human light and heavy chains as well as constant regions, produced by means of any of the known standard methods. The human antibody may include amino acid residues not encoded by human germline immunoglobulin sequences (e.g., mutations introduced by random or site-directed specific mutagenesis in vitro or by somatic mutation in vivo).

The antibodies used in the present invention can also be caninized or felinized.

“Caninized” forms of non-canine (e.g., murine) antibodies are genetically engineered antibodies that contain minimal sequence derived from non-canine immunoglobulin. Caninized antibodies are canine immunoglobulin sequences (recipient antibody) in which hypervariable region residues of the recipient are replaced by hypervariable region residues from a non-canine species (donor antibody) such as mouse having the desired specificity, affinity, and capacity. In some instances, framework region (FR) residues of the canine immunoglobulin sequences are replaced by corresponding non-canine residues. Furthermore, caninized antibodies may include residues that are not found in the recipient antibody or in the donor antibody. These modifications are made to further refine antibody performance. In general, the caninized antibody will include substantially all of at least one, and typically two, variable domains, in which all or substantially all of the hypervariable regions correspond to those of a non-canine immunoglobulin sequence and all or substantially all of the FRs are those of a canine immunoglobulin sequence. The caninized antibody optionally also will comprise a complete, or at least a portion of an immunoglobulin constant region (Fc), typically that of a canine immunoglobulin sequence

By the term “felinized antibody” is meant a feline antibody which contains minimal sequence derived from non-feline (e.g., mouse or human) immunoglobulin. In non-limiting examples, felinized antibodies are feline antibodies (recipient antibody) in which hypervariable region residues of the recipient antibody are replaced by hypervariable region residues from a non-feline species antibody (donor antibody), e.g., mouse, rat, rabbit, or human antibody having the desired specificity, affinity, and capacity. In some embodiments, the Fv framework residues of the feline immunoglobulin are replaced by corresponding non-feline residues. In some embodiments, felinized antibodies may contain residues which are not found in the recipient antibody or in the donor antibody. These modifications can be made to further refine antibody performance. In some embodiments, the felinized antibody will contain substantially all of at least one, and typically two, variable domains, in which all or substantially all of the hypervariable loops (complementary determining regions) correspond to those of a non-feline immunoglobulin and all or substantially all of the framework regions are those of a feline immunoglobulin sequence. The felinized antibody can also contain at least a portion of an immunoglobulin constant region (Fc), typically, that of a feline immunoglobulin. Felinized antibodies can be produced by molecular biology methods known in the art.

In a preferred embodiment, the antibody that binds specifically to the S100A4 protein or a fragment thereof has anti-fibrotic activity.

The term “anti-fibro”, “anti-fibrotic” or “anti-fibrosis” refers to the reparation and/or prevention of pathological polymerization of collagen in lung fibrosis, prostatic hypertrophy, keloid, myocarditis, collagen disease, etc. and reparation as well as normalization of the existing pathological fibrotic tissues.

The expression “having anti-fibrotic activity”, as used herein, refers to the ability of the antibodies to inhibit or reduce S100A4-induced fibrosis.

The “anti-fibrotic” treated by the present invention and as used herein refers to the ability of the antibodies to (1) prevent an excessive pathologic accumulation of collagenous scar or connective tissue in various body structures and organs (usually triggered by some injury, allergy, infection, or by some inherited genetic aberration), or (2) cause the non-surgical removal or biological dissolution of an existing excessive and pathologic accumulation of fibrotic collagenous tissue.

The synthesis of various collagens found in scar or fibrotic structures takes place in fibroblast cells which then extrude the collagen into the surrounding matrix. During this wound repair process, there are (1) a rapid proliferation and increase in the number of fibroblasts at the site, and (2) a sharp rise in the rate of the synthesis and extrusion of collagen. If these two phenomena are not prevented, the pathologic and progressive accumulation of collagen would cause polymerization and fibrotic disease (for example, impairment of respiratory function, impaired circulatory function via fibrotic changes in arterial walls, fibrotic degeneration of renal and liver function, degenerative musculoskeletal function, fibrotic degeneration of cardiac muscle or skeletal muscle, fibrotic degenerative changes in neuronal tissues in the central nervous system as well as the peripheral nervous system, etc.).

The anti-fibrotic activity can be determined in vitro by determining the ability of the antibody or fragment thereof to reduce the accumulation of extracellular matrix, investigating matrix proteins by IHC and/or as biomarkers in tissue samples and biofluid samples (some examples but not limited to include hydroxyproline, or collagen fibers from different subtypes). According to the present invention, an anti-S100A4 antibody is considered as being anti-fibrotic if it reduces at least 100%, at least 90%, at least 80%, at least 70%, at least 60%, at least 50%, at least 40%, at least 30%, at least 20% or at least 10% of the fibrotic activity of the S100A4 protein. Said anti-fibrotic activity can be evaluated by means of the assays described in Examples 1, 2 and 4 of the present invention.

In a preferred embodiment the antibody that binds specifically to the S100A4 protein or a fragment thereof produces a decrease of collagen fiber crosslinking. The decrease of collagen fiber crosslinking can be evaluated by means of the assay described in Examples 1 and 2 of the present invention, i.e., by staining the tissue with picrosirius red staining and visualizing the preparation under polarized light, as previously described (Junqueira L C, Bignolas G, Brentani R R. Picrosirius staining plus polarization microscopy, a specific method for collagen detection in tissue sections. Histochem J 1979; 11: 447-455).

In a preferred embodiment the antibody that binds specifically to the S100A4 protein or a fragment thereof produces a decrease in the expression of fibrosis biomarkers selected from the group consisting of BNP, SktAct, βMHC, Col1a1, Col3a1 and periostin. The decrease in the expression of fibrosis biomarkers can be evaluated by means of the assay described in Example 4 of the present invention, i.e., by measuring these biomarkers by quantitative RT-PCR.

In another embodiment the antibody that binds specifically to the S100A4 protein or a fragment thereof produces a decrease in the left ventricle (LV) hypertrophy in cardiac fibrosis. The decrease in the LV hypertrophy in cardiac fibrosis can be evaluated by means of the assay described in Examples 3 and 4 of the present invention, i.e., through the measurement of LV weight (mg) and normalized to the tibial length (mm).

In another embodiment the antibody that binds specifically to the S100A4 protein or a fragment thereof produces a decrease in the levels of S100AA protein. The decrease in the levels of S100A4 protein can be evaluated by means of the assay described in Examples 3 and 4, i.e., by measuring the levels of S100A4 by quantitative RT-PCR.

In another embodiment the antibody that binds specifically to the S100A4 protein or a fragment thereof produces a decrease in the levels of RAGE protein. The decrease in the levels of RAGE protein can be evaluated by means of the assay described in Examples 3 and 4, i.e., by measuring the levels of RAGE by quantitative RT-PCR.

The decrease of collagen fiber crosslinking, the decrease in the expression of fibrosis biomarkers, the decrease in the LV hypertrophy, the decrease in the levels of S100A4 protein or the decrease in the levels of RAGE protein can be confirmed by assays such as those disclosed in the examples of the present invention. The term “decrease” means that the collagen fiber crosslinking, the expression level of fibrosis biomarkers, the LV weight (mg)/tibial length (mm) ratio, the levels of S100A4 protein or of RAGE are lower than a reference value, particularly are lower than that of the subject when is not treated with the antibody. It is considered to be lower when it is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, or more lower than its reference value, than the level of the untreated subject.

The antibody fragments of the present invention preserve the capacity for binding to the S100A4 antigen of the whole antibody from which they derive and they also preserve the function of inhibiting the fibrotic activity of the S100A4 protein, preferably they preserve the anti-fibrotic activity of the whole antibody from which they derive.

The term “preserve the anti-fibrotic activity of the whole antibody from which they derive”, as used herein, refers to the ability of the antibody fragment to show substantially the anti-fibrotic activity of the complete antibody. An antibody fragment preserves the anti-fibrotic activity of the antibody if it shows at least 100%, 99%, 98%, 97%, 96%, 95%, 94%, 93%, 92%, 91%, 90%, 85%, 80%, 75%, 70%, 65%, 60%, 55% or 50% of the activity of the antibody.

The antibody or fragment thereof of the invention binds specifically to the S100A4 protein.

In a preferred embodiment, the antibodies specifically bind to human or murine S100A4 and not to the other S100 family proteins.

The antibodies useful in the invention must be specific for the S100A4 protein. The term “specific” refers to the capacity of the antibodies for binding specifically to the S100A4 protein and not to other proteins of the S100 family.

The phrase “specifically binds” when referring to antibodies and antigen binding fragments thereof means that the antibody binds S100A4, preferably murine, canine, feline, or human S100A4, with no or insignificant binding to other proteins, preferably to other proteins of the S100 family, more preferably to other murine or human proteins. The term however does not exclude the fact that antibodies of the invention may also be cross-reactive with other forms of S100A4. The phrase “specifically binds to” refers to a binding reaction that is determinative of the presence of the protein in a heterogeneous population of proteins and other biologics. Thus, under designated assay conditions, the specified binding moieties bind preferentially to a particular target protein and do not bind in a significant amount to other components present in a test sample. Specific binding to a target protein under such conditions may require a binding moiety that is selected for its specificity for a particular target antigen. A variety of assay formats may be used to select ligands that are specifically reactive with a particular protein. For example, solid-phase ELISA immunoassays, immunoprecipitation, Biacore, and Western blot are used to identify peptides that specifically react with S100A4, particularly with human or murine S100A4. Typically a specific or selective reaction will be at least twice background signal or noise and more typically more than 10 times background. Typically, the antibody binds with an association constant (Ka) of at least about 1×10⁶ M⁻¹ or 10⁷ M⁻¹, or about 10⁸ M⁻¹ to 10⁹ M⁻¹, or about 10¹⁰ M⁻¹ to 10¹¹ M⁻¹ or higher, and binds to the predetermined antigen with an affinity that is at least two-fold greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen”. Said expression includes antibodies previously disclosed in the prior art.

The term “high affinity” for an IgG antibody refers to an equilibrium association constant (Ka) of at least about 10⁷M⁻¹, at least about 10⁸M⁻¹, at least about 10⁹M⁻¹, at least about 10¹⁰M⁻¹, at least about 101M⁻¹, or at least about 10¹²M⁻¹ or greater, e.g., up to 10¹³M⁻¹ or 10⁴M⁻¹ or greater. However, “high affinity” binding can vary for other antibody isotypes. In a preferred embodiment the antibody is an IgG antibody, preferably an IgG1 antibody.

The term “Ka”, as used herein, is intended to refer to the equilibrium association constant of a particular antibody-antigen interaction. This constant has units of 1/M.

To identify the antibodies with the desired specificity, immunochemical assays, such as immunofluorescence, flow cytometry, Western blot and ELISA assays, radioimmunoassays, immunohistochemical assays, immunoprecipitations, or other immunochemical assays known in the art can be used. A number of protocols for competitive binding or immunoradiometric assays are known in the state of the art. Said immunoassays typically involve measuring the formation of a complex between an antibody and an immunogen of the S100A4 protein.

In a preferred embodiment the antibody for use according to the invention recognizes an epitope of S100A4 comprising the sequence ELPSFLGKRT (SEQ ID NO: 1), or an epitope of S100A4 comprising the sequence EGFPDKQPRKK (SEQ ID NO: 2) or is the antibody produced by the hybridoma ECACC 11051804. In another embodiment the antibody for use according to the invention recognizes an epitope of S100A4 consisting of SEQ ID NO: 1 or an epitope of S1004 consisting of SEQ ID NO: 2 or is the antibody produced by the hybridoma ECACC 11051804.

The epitope can be formed by the entire sequence SEQ ID NO: 1 or SEQ ID NO: 2 or by some amino acids of said sequence.

The expression “antibody that recognizes an epitope of S100A4” indicates that the antibody is capable of showing specific binding to the epitope without showing substantial binding to other epitopes not comprising this sequence. Suitable means for determining whether an antibody is capable of specifically binding to an epitope are well known in the art, wherein peptides representing the complete sequence of the target protein are tested against the antibody or fragment thereof. An antibody is considered to bind specifically to a given epitope if it binds to a peptide comprising the sequence of the epitope with substantially higher affinity than to a peptide which does not comprise the sequence of said epitope. The term “substantially higher affinity”, as used herein, refers to an affinity level for a particular amino acid sequence which is distinguishable from a level of other amino acid sequence when detected with an intended measurement device or method. Preferably, the affinity of the binding between the antibody and peptide comprising the epitope is at least one order of magnitude higher, at least two orders of magnitude, at least three orders of magnitude higher, at least four orders of magnitude, at least five orders of magnitude higher, at least six orders of magnitude higher than the affinity of the binding between the antibody and a peptide which does not comprise the sequence of the epitope. The association constant (Ka) of binding with substantially high affinity is, for example, at least 10⁷M⁻¹, preferably at least 10⁸M⁻¹, and more preferably at least 10⁹M⁻¹ or lower.

In a preferred embodiment, the CDRs of the light chain of the variable region of the antibody against S100A4 comprise the sequences set forth in SEQ ID NO: 3, 4 and 5, or a functionally equivalent variant thereof, and the CDRs of the heavy chain of the variable region comprise the sequences set forth in SEQ ID NO: 6, 7, 8 or a functionally equivalent variant thereof. In another embodiment, the CDRs of the light chain of the variable region of the antibody against S100A4 consist of the sequences set forth in SEQ ID NO: 3, 4 and 5, or a functionally equivalent variant thereof, and the CDRs of the heavy chain of the variable region consist of the sequences set forth in SEQ ID NO: 6, 7, 8 or a functionally equivalent variant thereof. SEQ ID NO: 3 to SEQ ID NO: 8 correspond to the sequences of the CDRs of the antibody produced by the hybridoma cell line deposited with the accession number ECACC 10022401 (Table 1).

TABLE 1
CDRs sequences of the antibody produced by the
hybridoma cell line deposited with the accession
number ECACC 10022401.

| SEQ ID NO: | Region | Sequence |
|---|---|---|
| 3 | CDRL1 | RSSQSIVHSNGNTYLE |
| 4 | CDRL2 | KVSNRLS |
| 5 | CDRL3 | FQGSHVPFT |
| 6 | CDRH1 | ETYMH |
| 7 | CDRH2 | RIDPANGNTKDDPKFQG |
| 8 | CDRH3 | SYAMDY |

The acronym “CDRs” refers to the complementarity determining region amino acid sequences of an antibody which are the hypervariable domains of immunoglobulin heavy and light chains. See, e.g., Kabat et al., Sequences of Proteins of Immunological Interest, 4th Ed., U.S. Department of Health and Human Services, National Institutes of Health (1987). There are three heavy chain and three light chain CDRs (or CDR regions) in the variable portion of an immunoglobulin. Thus, “CDRs”, as used herein, refers to all three heavy chain CDRs, or all three light chain CDRs (or both all heavy and all light chain CDRs, if appropriate). The structure and protein folding of the antibody may mean that other residues are considered part of the antigen binding region and would be understood to be so by a skilled person. See for example Chothia et al., (1989) Conformations of immunoglobulin hypervariable domains; Nature 342, p877-883. CDRs provide the majority of contact residues for the binding of the antibody to the antigen or epitope. CDRs of interest in this invention are derived from donor antibody variable heavy and light chain sequences, and include analogs of the naturally occurring CDRs, which analogs also share or retain the same antigen binding specificity and/or neutralizing ability as the donor antibody from which they were derived.

Throughout this specification, amino acid residues in antibody sequences are numbered according to the Kabat scheme. Similarly, the terms “CDR”, “CDRL1”, “CDRL2”, “CDRL3”, “CDRH1”, “CDRH2”, “CDRH3” follow the Kabat numbering system as set forth in Kabat et al; Sequences of proteins of Immunological Interest NIH, 1987. It will be apparent to those skilled in the art that there are alternative definitions of CDR sequences such as for example those set out in Chothia et al. (1989).

In another preferred embodiment, the FR region of the light chain of the variable region of the antibody against S100A4 comprises the sequences set forth in SEQ ID NO: 9, 10, 11 and 12 or a functionally equivalent variant thereof, and the FR region of the heavy chain of the variable region of the antibody against S100A4 comprises the sequences set forth in SEQ ID NO: 13, 14, 15 and 16 or a functionally equivalent variant thereof. In another embodiment, the FR region of the light chain of the variable region of the antibody against S100A4 consists of the sequences set forth in SEQ ID NO: 9, 10, 11 and 12 or a functionally equivalent variant thereof, and the FR region of the heavy chain of the variable region of the antibody against S100A4 consists of the sequences set forth in SEQ ID NO: 13, 14, 15 and 16 or a functionally equivalent variant thereof. SEQ ID NO: 9 to SEQ ID NO: 16 correspond to the sequences of the FRs of the antibody produced by the hybridoma cell line deposited with the accession number ECACC 10022401 (Table 2).

TABLE 2
FRs sequences of the antibody produced by the hybridoma cell
line deposited with the accession number ECACC 10022401.

| SEQ ID NO: | Region | Sequence |
|---|---|---|
| 9 | FRL1 | DVLMTQTPLSLPVSLGDQASISC |
| 10 | FRL2 | WYLQKTGQSPELLIY |
| 11 | FRL3 | GVPDRFSGSGSGTDFTLKISRVEAEDLGVYY |
| | | C |
| 12 | FRL4 | FGSGTKLEIK |
| 13 | FRH1 | EAQLQQSGAELVKPGASVKLSCTASGFNIQ |
| 14 | FRH2 | WVKQRPEQGLEWIG |
| 15 | FRH3 | KASITVDTSSNTAYLQLSSLTSEDTAVYYCAS |
| 16 | FRH4 | WGQGTSVTVSS |

“FR” as used herein relates to the framework region

In another preferred embodiment, the antibody against S100A4 comprises at least a VL region comprising the sequence set forth in SEQ ID NO: 17 or a functionally equivalent variant thereof, and at least a VH region comprising the sequence set forth in SEQ ID NO: 18 or a functionally equivalent variant thereof. In another embodiment, the antibody against S100A4 comprises at least a VL region consisting of the sequence set forth in SEQ ID NO: 17 or a functionally equivalent variant thereof, and at least a VH region consisting of the sequence set forth in SEQ ID NO: 18 or a functionally equivalent variant thereof. SEQ ID NO: 17 and 18 correspond, respectively, to the sequences of the VL region and the VH region of the antibody produced by the hybridoma cell line deposited with the accession number ECACC 10022401 (Table 3).

TABLE 3
VL and VH sequences of the antibody produced by the hybridoma cell
line deposited with the accession number ECACC 10022401.

| SEQ ID NO: | Region | Sequence |
|---|---|---|
| 17 | VL | DVLMTQTPLSLPVSLGDQASISCRSSQSIVHSNGNTYL |
| | | EWYLQKTGQSPELLIYKVSNRLSGVPDRFSGSGSGTD |
| | | FTLKISRVEAEDLGVYYCFQGSHVPFTFGSGTKLEIK |
| 18 | VH | EAQLQQSGAELVKPGASVKLSCTASGFNIQETYMHWV |
| | | KQRPEQGLEWIGRIDPANGNTKDDPKFQGKASITVDTS |
| | | SNTAYLQLSSLTSEDTAVYYCASSYAMDYWGQGTSVT |
| | | VSS |

The term “VL region” refers to the variable region of the light chain of the antibody; whereas the term “VH region” refers to the variable region of the heavy chain of the antibody.

In a preferred embodiment, the antibody against S100A4 comprises the sequence SEQ ID NO: 17 and the sequence SEQ ID NO: 18.

A “functionally equivalent variant” of the sequence of the antibody is a sequence that when forming part of an antibody against S100A4 or a binding fragment thereof preserves the anti-fibrotic activity of said antibody and has a minimal identity in the amino acid sequence.

In general, modifications in the amino acid sequence of the antibody of the disclosure are also contemplated. For example, it can be desirable to improve the binding affinity and/or other biological properties of the antibody. The variants of the amino acid sequences of the antibody are prepared by introducing the suitable nucleotide changes in the nucleic acid encoding the antibody, or by means of peptide synthesis. Said modifications include, for example, eliminations and/or insertions and/or substitutions (such as conservative substitutions) of one or more residues in the amino acid sequences of the antibody. Any combination of elimination, insertion and substitution is performed to achieve the final construct, provided that the final construct has the desired characteristics, i.e., S100A4 binding specificity and antagonist anti-fibrotic activity of said protein. The changes in the amino acids can also alter the post-translational processes of the antibody, such as changing the number or the position of the sites of glycosylation.

Some insertions in the amino acid sequence include amino terminus and/or carboxy terminus fusions varying in length from one residue up to polypeptides containing one hundred or more residues, as well as insertions within the sequence of one or several amino acid residues. Some examples of terminal insertions include an antibody with an N-terminus methionyl residue, or the antibody fused to a cytotoxic polypeptide. Other variants by insertion of the antibody molecule include fusion with the N- or C-terminus of the antibody of an enzyme, or a polypeptide increasing the serum half-life of the antibody. Another type of variant is a variant by amino acid substitution. These variants have at least one amino acid residue of the antibody substituted with a different residue. The sites of major interest for mutagenesis by antibody substitution include the hypervariable regions, but alterations in the FR are also contemplated.

The functionally equivalent variants of the sequences of the antibodies that are involved in the binding to the S100A4 protein must conserve their capacity for binding to the S100A4 protein and also the capacity for inhibiting the fibrotic function of S100A4 protein. Said function can be checked by means of the methods known by the skilled in the art, for example by means of the assays as described in Example 2 and 4 of the present application.

The functionally equivalent variants of the antibody sequences of the invention include polypeptides showing at least 60%, 65%, 70%, 72%, 74%, 76%, 78%, 80%, 90%, 95%, 97%, 98%, 99% identity with the polypeptide sequences mentioned before; preferably having at least 98% identity. The degree of identity between two polypeptides is determined as described previously in relation to the variants of S100A4.

In another preferred embodiment, the antibody against S100A4 is a monoclonal antibody produced by the hybridoma cell line deposited with the accession number ECACC 10022401.

In another preferred embodiment, the antibody against S100A4 is a monoclonal antibody produced by the hybridoma cell line deposited with the accession number ECACC 11051801, ECACC 11051802, ECACC 11051803, or ECACC 11051804.

In the context of the present invention, “hybridoma” is understood as the product of the fusion of a B-cell clone descendent of a single unique stem cell, and of a myeloma cell. The expression “hybridoma cell line” refers to a cell line formed by hybridomas as previously defined.

The European Collection of Cell Cultures (ECACC) has assigned to hybridomas 5C3-1B8-1F4, 6B9-1E8-2A8, 5A3-4A6-5B6, 1E2-2H4-2G8 and 8B6-2F6-1H9-1H10 the respective deposit numbers ECACC 10022401, ECACC 11051801, ECACC 11051802, ECACC 11051803 and ECACC 11051804. In the present document, hybridomas 5C3-1B8-1F4, 6B9-1E8-2A8, 5A3-4A6-5B6, 1E2-2H4-2G8 and 8B6-2F6-1H9-1H10 and the antibodies produced by said hybridomas are indicated by means of their abbreviated name 5C3, 6B9, 5A3, 1E2 and 8B6, respectively. Said hybridoma cell lines were obtained by standard methodologies. Briefly, mice were immunized with a human recombinant S100A4 protein of SEQ ID NO: 20:

GSHMACPLEK ALDVMVSTFH KYSGKEGDKF KLNKSELKEL
LTRELPSFLG KRTDEAAFQK LMSNLDSNRD NEVDFQEYCV
FLSCIAMMCN EFFEGFPDKQ PRKK

Then, cells were extracted from the spleen of the immunized mouse which were fused with myeloma cells in the presence of a fusion inducer such as PEG-1500. The hybridomas were selected in HAT medium and each selected clone was subcloned by limiting dilution. The clones suitable for expansion were adapted to the DMEM/F12 medium and were frozen, constituting the hybridoma cell lines. The procedure for obtaining these hybridoma cell lines is disclosed in EP 2 580 240 B1.

Hybridoma 5C3-1B8-1F4 was deposited on 24 Feb. 2010 in the European Collection of Cell Cultures (ECACC) (Porton Down, Salisbury, SP4 OJG, United Kingdom) under the conditions stipulated in the Budapest Treaty. Hybridomas 6B9-1E8-2A8, 5A3-4A6-5B6, 1E2-2H4-2G8 and 8B6-2F6-1H9-1H10 were deposited on 18 May 2011 in the European Collection of Cell Cultures (ECACC), Porton Down, Salisbury, SP4 OJG, United Kingdom, as a legally recognized institution for that purpose in accordance with the Budapest Treaty, of 28 Apr. 1997, on the International Recognition of the Deposit of Microorganisms. The depositors were Francesc Mitjans and Marc Masa from Leitat Technological Center with address Baldiri Reixach 15-21 Helix Building, Barcelona, 08028, Spain. The culture conditions of said hybridoma lines which allow obtaining the anti-S100A4 antibodies are typical conditions known by the persons skilled in the art. In a preferred embodiment, the medium suitable for the culture of said cells is a medium comprising DMEM/F12 and L-glutamine. The conditions in which said culture are performed are preferably in a humid environment and at a temperature of 37° C. with standard air atmosphere or 5% CO₂ enriched air.

Cross-reactivity characterization of monoclonal antibodies 5C3, 1E2, 6B9, 8B6, and 5A3, immune-characterizations of 5C3 by Western-blot and immunohistochemistry, and epitope determination of monoclonal antibodies 5C3, 1E2, 6B9, 8B6 and 5A3 were disclosed in EP 2 580 240 B1.

The antibodies or fragments thereof for the medical use of the invention are administered in a therapeutically effective amount.

“Therapeutically effective amount” means the amount of an active substance that, when administered to a subject for treating a disease, disorder, or other undesirable medical condition, is sufficient to have a beneficial effect with respect to that disease, disorder, or condition. The therapeutically effective amount will vary depending on the chemical identity and formulation form of the active substance, the disease or condition and its severity, and the age, weight, and other relevant characteristics of the patient to be treated. Determining the therapeutically effective amount of a given active substance is within the ordinary skill of the art and typically requires no more than routine experimentation.

The antibodies and fragments of the invention can be used in any subject. “Subject”, as used herein, relates to any animal classified as mammal and includes but is not limited to domestic and farm animals, primates and humans, for example human beings, non-human primates, cows, horses, pigs, sheep, goats, dogs, cats or rodents. Preferably, the subject is a female or male human being of any race or age. In the context of the present invention, the subject is a subject who potentially suffers from a pathogenic fibrosis. In another embodiment, the subject is a dog. In another embodiment the subject is a cat.

The antibodies and fragments of the invention can be administered by any type of suitable route, such as by oral route, topical route, by inhalation or parenteral route; preferably by parenteral route. The antibodies and fragments of the invention can be formulated as pharmaceutical compositions including pharmaceutically acceptable excipients necessary for the formulation of the desired dosage form. Subcutaneous, intramuscular and intravenous route are generally preferred. The preferred route of administration is the endovenous route.

### Diagnostic Method

In another aspect, the invention relates to an in vitro method for diagnosing a disease associated to pathogenic fibrosis in a subject which comprises:
a) Detecting the levels of the S100A4 protein or of a variant thereof in a sample of said subject, and
b) Comparing said levels with a reference value,
   wherein increased levels of the S100A4 protein or of a variant thereof with respect to the reference value are indicative of the subject suffering from a disease associated to pathogenic fibrosis.

“In vitro method for diagnosing a disease associated to pathogenic fibrosis”, as used herein, relates to a method which allows showing the existence of a disease associated to pathogenic fibrosis in a subject by means of detecting the presence of S100A4 protein or of a variant thereof in a sample from the subject. As the skilled in the art will understand, such evaluation may not be correct for 100% of the subjects to be diagnosed, although it preferably is. The term, however, requires being able to identify a statistically significant part of the subjects.

In another aspect, the invention relates to an in vitro method for diagnosing and treating a disease associated to pathogenic fibrosis in a subject which comprises:
a) detecting the levels of the S100A4 protein or of a variant thereof in a sample of said subject,
b) comparing said levels with a reference value, and
c) administering a therapy suitable for treating fibrosis to the subject showing increased levels of the S100A4 protein or of a variant thereof with respect to the reference value.

“Therapy suitable for treating fibrosis”, as used herein, relates to any medicament capable of reducing the formation of excess fibrous connective tissue in an organ or tissue and/or reducing collagen fiber crosslinking or the expression of one or more fibrosis biomarkers selected from the group consisting of BNP, SktAct, βMHC, Col1a1, Col3a1 and periostinin in a tissue.

Illustrative, non-limitative examples of drugs suitable for treating fibrosis, are resveratrol, metformin, pirfenidone, nintedanib, C75 GW4064, MET409, exenatide, hesperidin, PBI-4050, 2-DG, 3-Bromopyruvate, WZB-117, pravastatin, tipelukast, 3PO, pentoxifylline, omacor, dichloroacetate, shikonin, IVA337, ciprofibrate, fenofibrate, WY-14643, troglitazone, elafibranor, saracatinib, 15d-PGJ2, ciglitazone, caffeic acid, caffeine plus chlorogenic acid, G1262570, pioglitazone, rosiglitazone, liothyronine or pioglitazone.

“Disease associated to pathogenic fibrosis” has been previously defined and is equally applicable to this aspect of the invention.

In a preferred embodiment, the disease associated to pathogenic fibrosis is associated to increased levels of S100A4.

The expression “pathogenic fibrosis associated to increased levels of S100A4” has been previously defined and is equally applicable to this aspect of the invention.

In another preferred embodiment the disease is not silicon-induced fibrosis, asbestos-induced fibrosis or cystic fibrosis.

In another preferred embodiment, the disease is not associated to metastasis.

In another preferred embodiment, the disease is selected from the group consisting of liver fibrosis, cardiac fibrosis, kidney fibrosis and lung fibrosis. In a more preferred embodiment, the disease is cardiac fibrosis. In another more preferred embodiment, the disease is liver fibrosis.

“Subject”, as used herein, relates to any animal classified as mammal and includes but is not limited to domestic and farm animals, primates and humans, for example human beings, non-human primates, cows, horses, pigs, sheep, goats, dogs, cats or rodents. Preferably, the subject is a female or male human being of any race or age. In the context of the present invention, the subject is a subject who potentially suffers from a pathogenic fibrosis. In another embodiment, the subject is a dog. In another embodiment the subject is a cat.

The first step of the diagnostic method of the invention comprises detecting the levels of the S100A4 protein or of a variant thereof in a sample of said subject.

The term “protein”, as used herein, refers to a molecular chain of amino acids, joined by covalent or non-covalent bonds. The term further includes all the physiologically relevant post-translational chemical modification forms. Post-translational modifications which fall within the scope of the present invention include, for example, signal peptide cleavage, glycosylation, acetylation, phosphorylation, isoprenylation, proteolysis, myristoylation, protein folding and proteolytic process, etc. Additionally, the proteins can include non-natural amino acids formed by post-translational modifications or by means of introducing non-natural amino acids during translation.

The term “S100A4” has been defined in the context of the first inventive aspect of the invention. For the diagnostic method of the invention, the detected S100A4 is that which corresponds to the species to which the subject from which the sample to be analyzed has been extracted belongs.

As mentioned above, variants of said protein can also be used to measure the levels of the S100A4 protein in the method of the invention.

Therefore, variants of the S100A4 protein can be: (i) those in which one or more of the amino acid residues are substituted by a conserved or non-conserved amino acid residue (preferably a conserved amino acid) and such substituted amino acid residue may or may not be encoded by the genetic code, (ii) those in which there are one or more modified amino acid residues, e.g. residues that are modified by the coupling of substituting groups, (iii) those in which the protein is an alternative splicing variant of the S100A4 and/or (iv) fragments of the protein. The fragments include proteins generated through proteolytic process (including proteolysis at multiple sites) of an original sequence. Said variants fall within the scope of the present invention.

Variants according to the present invention include amino acid sequences that are at least 60%, 65%, 70%, 75%, 80%, 85%, 90%, 95%, 96%, 97%, 98% or 99% similar or identical to the original amino acid sequence. As it is known, the “similarity” between two proteins is determined by means of comparing the amino acid sequence of a protein with a sequence of a second protein. The degree of identity between two proteins is determined using computer algorithms and methods that are widely known by the person skilled in the art, preferably using the BLASTP algorithm [BLASTManual, Altschul, S., et. al., NCBI NLM NIH Bethesda, Md. 20894, Altschul, S., et. al., J. Mol. Biol. 215: 403-410 (1990)].

In a particular embodiment, the variant is a variant from mammal, preferably a human variant, more preferably with at least 60%, 70%, 80%, 90%, 95% or 96% similarity or identity with the original amino acid sequence.

The person skilled in the art will appreciate that the method of the invention can be put into practice using both the absolute level and the relative level of expression of the S100A4 protein. Thus, in the present invention, the expression “levels of the S100A4 protein” is used to refer both the absolute levels and the relative levels of said protein.

The expression “absolute levels” refers to the total amount of the protein of interest in a sample. Said value may be given as the concentration of protein expressed in units of mass per unit of volume (e.g. in ng/ml of sample), in the number of protein molecules per unit of volume (e.g. in pmol protein/ml of sample), in the units of mass of S100A4 protein per unit of mass of total protein (pg S100A4/mg total protein) or in the number of S100A4 molecules per unit of mass of total protein (e.g. in pmol S100A4/mg of total protein).

The expression “relative levels” refers to the relationship between the levels of expression of the S100A4 protein object of the study and of a reference protein, i.e., the concentration of S100A4 protein in normalized form with respect to said reference protein is defined.

In order to normalize the values of protein between the different samples, it is possible to compare the levels of S100A4 protein in the samples to be analyzed with the expression of a control protein. “Control protein” in the present invention is understood as a protein the levels of expression of which do not change or only change in limited amounts in the tumor cells with respect to the non-tumor cells. Preferably, the control protein is a protein encoded by genes that are constitutively expressed, that are those genes always active or being transcribed constantly, such that these proteins are constitutively expressed and carry out essential cellular functions. Preferred control proteins that can be used in the present invention include, without limitation, β-2-microglobulin (B2M), ubiquitin, 18-S ribosomal protein, cyclophilin, GAPDH, PSMB4, tubulin and actin.

The person skilled in the art understands that mutations in the amino acid sequence of the S100A4 protein do not affect the detection of the expression thereof and, therefore, the variants of this protein generated by mutations of the amino acid sequence fall within the scope of the present invention.

The levels of expression of the S100A4 protein can be detected and quantified by means of conventional methods. Said methods include, without limitation, the detection of S100A4 by measuring its affinity to one of its ligands such as RAGE, and the subsequent quantification of the S100A4-ligand complex; or by means of using an antibody against S100A4, such as those disclosed in relation to the previous aspect of the invention, for example the monoclonal antibodies with capacity of binding specifically to the S100A4 protein (or fragments thereof which contain the antigenic determinants) produced by a hybridoma selected from the group consisting of ECACC 10022401, ECACC 11051801, ECACC 11051802, ECACC 11051803 and ECACC 11051804 or a functional variant of said antibody. Then, the resulting antigen-antibody complexes are quantified.

The invention also contemplates the use of functional variants of said antibodies. “Functional variant” of the monoclonal antibodies of the invention is understood as any molecule sharing with said monoclonal antibodies one or more of the functions described in the present invention associated with said monoclonal antibodies, both in vitro and in vivo, and having a minimal identity in the amino acid sequence. The functional variants of the monoclonal antibodies of the invention can be derived from said sequences by means of insertion, substitution or deletion of one or more amino acids and can be obtained by recombinant and/or synthetic means.

The functional variants of the monoclonal antibodies of the invention must conserve their capacity for binding to the S100A4 antigen and also the capacity for inhibiting one or more characteristic functions of the S100A4 protein, such as the fibrosis. Said functions can be determined by means of the methods described in the examples of the present invention.

The functional variants of the monoclonal antibodies of the invention include polypeptides showing at least 60%, 65%, 70%, 72%, 74%, 76%, 78%, 80%, 90%, 95%, 97%, 99% similarity or identity with the polypeptide sequence of said antibodies. The degree of identity between two polypeptides is determined using algorithms implemented in a computer and methods which are widely known by the persons skilled in the art. The identity between two amino acid sequences is preferably determined using the BLASTP algorithm (BLAST Manual, Altschul, S. et al., NCBI NLM NIH Bethesda, Md. 20894, Altschul, S., et al., J., 1990, Mol. Biol. 215:403-410).

There is a wide variety of well known assays which can be used in the present invention, these assays use primary non-labeled antibodies and secondary labeled antibodies: such techniques include Western-blot or Western transfer, ELISA (Enzyme Linked Immunosorbent Assay), RIA (radioimmunoassay), competitive EIA (competitive enzyme immunoassay), DAS-ELISA (double antibody sandwich ELISA), or techniques based on the use of protein microarrays or biochips which include specific antibodies or assays based on the colloidal precipitation in forms such as reactive strips. Other ways for detecting the S100A4 protein include techniques such as affinity chromatography, ligand binding assays, etc.

In a particular embodiment, the quantification of the levels of S100A4 is performed by means of Western-blot or ELISA.

In yet a more particular embodiment, the levels of the S100A4 protein or of its variants are determined by Western-blot. Western-blot is based on detecting the previously resolved proteins by means of electrophoresis in gel under denaturing conditions and being immobilized on a membrane, generally nitrocellulose, by means of incubation with an antibody specific for S100A4 and a development system (e.g. chemiluminescent).

In another preferred embodiment, the diagnostic is performed by means of ELISA. Said technique is based on the detection of the S100A4 protein in a sample by means of an anti-S100A4 antibody immobilized on a substrate and the subsequent detection of the S100A4-antibody complex by means of a second antibody.

In a preferred embodiment, the levels of the S100A4 protein or of a variant thereof are detected by means of using an antibody or a fragment thereof that binds specifically to the S100A4 protein.

The term “antibody against S100A4” and preferred embodiments have been disclosed in relation to the medical use and are equally applicable to this aspect of the invention.

In addition, the antibodies used in the method of the invention may or may not be labeled with a detectable agent. In a particular embodiment the antibody used is conjugated to a detectable agent.

In the context of the present invention, the terms “detectable agent” and “labeling” are synonyms and they refer to an agent the nature of which allows its detection by means of enzymatic, radioactive or fluorescence methods. The detectable compound can be an enzyme, a radioactively labeled compound or a radioactive isotope, a fluorochrome, a chemiluminescent reagent, an enzymatic substrate or cofactor, an enzymatic inhibitor, a particle, a dye, etc.

The compounds radioactively labeled by means of radioactive isotopes, also called radioisotopes or radionuclides, may include, without limitation, ³H, ¹⁴C ¹⁵N, ³⁵S, ⁹⁰Y, ⁹⁹Tc, ¹¹¹In, ¹²⁵I, ¹³¹I. The fluorescent labels may include, without limitation, rhodamine, phosphorus-lanthanides or FITC. The enzymatic labels may include, without limitation, horseradish peroxidase, β-galactosidase, luciferase or alkaline phosphatase. The preferred labeling include, but are not limited to, fluorescein, a phosphatase such as alkaline phosphatase, biotin, avidin, a peroxidase such as horseradish peroxidase and compounds related to biotin or compounds related to avidin (for example, streptavidin or ImmunoPure® NeutrAvidin available from Pierce, Rockford, IL).

In a more preferred embodiment, the antibody against S100A4 recognizes an epitope of S100A4 comprising the sequence ELPSFLGKRT (SEQ ID NO: 1), an epitope of S100A4 comprising the sequence EGFPDKQPRKK (SEQ ID NO: 2) or is the antibody produced by the hybridoma ECACC 11051804.

In a more preferred embodiment, the CDRs of the light chain of the variable region of the antibody against S100A4 comprise the sequences set forth in SEQ ID NO: 3, 4 and 5, or a functionally equivalent variant thereof, and the CDRs of the heavy chain of the variable region comprise the sequences set forth in SEQ ID NO: 6, 7, 8 or a functionally equivalent variant thereof.

In a more preferred embodiment, the FR region of the light chain of the variable region of the antibody against S100A4 comprises the sequences set forth in SEQ ID NO: 9, 10, 11 and 12 or a functionally equivalent variant thereof, and the FR region of the heavy chain of the variable region of the antibody against S100A4 comprises the sequences set forth in SEQ ID NO: 13, 14, 15 and 16 or a functionally equivalent variant thereof.

In another preferred embodiment, the antibody against S100A4 comprises at least a VL region comprising the sequence set forth in SEQ ID NO: 17 or a functionally equivalent variant thereof and at least a VH region comprising the sequence set forth in SEQ ID NO: 18 or a functionally equivalent variant thereof.

In another preferred embodiment, the antibody against S100A4 is a monoclonal antibody produced by the hybridoma cell line deposited with the accession number ECACC 10022401.

In another preferred embodiment, the antibody against S100A4 is a monoclonal antibody produced by the hybridoma cell line deposited with the accession number ECACC 11051801, ECACC 11051802, ECACC 11051803 or ECACC 11051804.

The method of the invention requires detecting the levels of the S100A4 protein or of a variant thereof in a sample of said subject.

The term “sample”, as used herein, encompasses a variety of sample types obtained from a subject and useful in the procedure of the invention. Biological samples may include, but are not limited to, solid tissue samples, liquid tissue samples, biological fluids, aspirates, cells and cell fragments. Specific examples of biological samples include, but are not limited to, solid tissue samples obtained by surgical removal, a pathology specimen, an archived sample, or a biopsy specimen, tissue cultures or cells derived therefrom and the progeny thereof, and sections or smears prepared from any of these sources. Biological samples also include any material derived from the body of a mammal, including, but not limited to, blood, cerebrospinal fluid, serum, plasma, urine, nipple aspirate, fine needle aspirate, tissue lavage such as ductal lavage, saliva, sputum, ascites fluid, liver, kidney, breast, bone, bone marrow, testes, brain, ovary, skin, lung, prostate, thyroid, pancreas, cervix, stomach, intestine, colorectal, brain, bladder, colon, uterine, semen, lymph, vaginal pool, synovial fluid, spinal fluid, head and neck, nasopharynx tumors, amniotic fluid, breast milk, pulmonary sputum or surfactant, urine, fecal matter and other liquid samples of biologic origin, and may refer to either the cells or cell fragments suspended therein, or to the liquid medium and its solutes.

In a preferred embodiment the sample is a biofluid, preferably a biofluid from affected organs. The term “biofluid” in the context of the present invention refers to any biological secretion or fluid, whether physiological or pathological, which is produced in the body of a subject. Such biofluids include, without limitation, blood, plasma, serum, bronchoalveolar washing fluid, urine, nasal secretion, ear secretion, urethral secretion, cerebrospinal fluid, pleural fluid, synovial fluid, peritoneal fluid, ascites fluid, pericardial liquid, amniotic fluid, gastric juice, lymphatic fluid, interstitial fluid, saliva, sputum, liquid deposition, tears, mucus, sweat, milk, semen, vaginal secretions, fluid coming from ulcer, blisters, abscesses and other surface eruptions. Said samples can be obtained by conventional methods, using processes known in the state of art by the person skilled in the art, such as blood extraction, instillation and aspiration of liquid during bronchofibroscopy, cisternal, ventricular or lumbar puncture, pleural puncture or thoracocentesis, joint or synovial percutaneous puncture, abdominal puncture, amniocentesis, expectoration, peritoneal percutaneous puncture, pericardial percutaneous puncture, etc., or by simple harvesting. Preferably the biofluid is selected from bronchoalveolar washing fluid, urine, pleural fluid, pericardial liquid or sputum.

In a preferred embodiment, the sample is blood, serum or plasma; preferably serum or plasma.

The diagnostic method of the invention comprises in a second step comparing the levels detected in step a) with a reference value.

The “reference value” is obtained from a sample collection formed preferably by a mixture of the sample to be analyzed from normal individuals not affected by a disease associated to pathogenic fibrosis. Said reference value can be determined by means of techniques well known in the state of the art, for example, determining the mean of the levels of S100A4 protein measured in a sample taken from healthy subjects. The reference value can also be obtained from the constitutively expressed proteins taken from the same subject to be analyzed.

Once the reference value is established, the value of the levels of S100A4 obtained in step (a) can be compared with this reference value and, therefore, allows detecting alterations in the levels of S100A4 protein of the subject with respect to the reference value. More specifically, in the method of the invention, an increase of the levels of S100A4 with respect to the reference value is indicative of the subject suffering from a pathogenic fibrosis.

In the context of the present invention, “increased levels” with respect to the reference value is understood as a variation of the levels of S100A4 above the reference value of at least 1.1 times, 1.5 times, 5 times, 10 times, 20 times, 30 times, 40 times, 50 times, 60 times, 70 times, 80 times, 90 times, 100 times or even more times as compared to the reference value.

All the embodiments disclosed in the context of the medical uses of the invention are equally applicable to the diagnostic method of the invention.

### Methods for Customizing Therapy

The diagnostic method of the invention is also useful for documenting the expression of S100A4 produced by a fibrotic tissue prior to administering S100A4 selecting drugs to allow a suitable selection of patients and the determination of the optimal dose.

Therefore, in another aspect, the invention relates to an in vitro method for designing a customized therapy for a subject diagnosed with a disease associated to pathogenic fibrosis which comprises:
a) detecting the levels of the S100A4 protein or of a variant thereof in a sample of said subject, and
b) comparing said levels with a reference value,
   wherein increased levels of the S100A4 protein or of a variant thereof with respect to the reference value are indicative that the subject is to be treated with an antibody or a fragment thereof that binds specifically to the S100A4 protein.

In this context, the “subject” is a subject who has been previously diagnosed with a disease associated with pathogenic fibrosis. It includes human and non-human animals. Non-human animals includes all vertebrates, e.g., mammals and non-mammals, such as primates and non-human primates, sheep, dog, rabbits, rats, mice, cow, chickens, amphibians, and reptiles.

In a preferred embodiment, the step (a) is carried out by means of using a monoclonal antibody produced by a hybridoma selected from the group consisting of ECACC 10022401, ECACC 11051801, ECACC 11051802, ECACC 11051803 and ECACC 11051804 or an S100A4 binding fragment thereof.

In a preferred embodiment, the patient is to be treated with an antibody selected from the group consisting of: an antibody that recognizes an epitope of S100A4 comprising the sequence ELPSFLGKRT (SEQ ID NO: 1), an antibody that recognizes an epitope of S100A4 comprising the sequence EGFPDKQPRKK (SEQ ID NO: 2) and the antibody produced by the hybridoma ECACC 11051804.

Preferably, the subject is selected for being treated with an anti-S100A4 antibody and the same antibody is used to monitor the levels of S100A4. When the levels of S100A4 protein are higher than a reference value, the subject is candidate for a therapy with the antibody.

The term “designing a customized therapy” means that the results obtained by said method are useful to decide if the subject is a candidate for a treatment with the antibodies of the invention.

The terms “disease associated to pathogenic fibrosis”, “S100A4 protein”, “levels”, “variant of said protein”, “reference value”, “increased”, “antibody”, “antibody fragment”, “binds specifically”, “biofluid”, “antibody”, “hybridoma”, and “functional variant of the antibody” have been defined previously.

All the particular embodiments of the medical uses and diagnostic methods are also applicable to this aspect.

### Uses of the Kits of the Invention

In another aspect, the invention relates to the use of a kit comprising an antibody or a fragment thereof that binds specifically to the S100A4 protein for diagnosing a disease associated with pathogenic fibrosis in a sample of a subject.

The term “kit”, as used in the present document, refers to a combination of a set of reagents suitable for detecting the levels of S100A4 together with one or more types of elements or components (for example, other types of biochemical reagents, containers, packaging suitable for its commercial sale, substrates to which the reagents are bound, electronic hardware components, etc.).

In the present invention “reagent suitable for detecting the levels of S100A4” is understood as a specific anti-S100A4 antibody or a polypeptide having at least one fragment of the sequence of said monoclonal antibody with capacity for binding to S100A4 and, optionally, reagents for detecting one or more constitutive proteins.

As it will be understood by the person skilled in the art, the antibodies of the kit of the invention can be used in all the techniques for determining the levels of protein known to be suitable for the analysis of a sample, such as Western-blot or Western transfer, ELISA, RIA, competitive EIA, DAS-ELISA, techniques based on the use of biochips, protein microarrays, assays of colloidal precipitation in reactive strips, etc.

The antibodies can be fixed to a solid support such as a membrane, a plastic or a glass, optionally treated to facilitate the fixation of said antibodies to the support. Said solid support comprises, at least, a set of antibodies which specifically recognize the S100A4 protein, and which can be used for detecting the levels of expression of said protein.

The kits of the invention can additionally comprise reagents for detecting a protein encoded by a constitutive gene. The availability of said additional reagents allows normalizing the measurements performed in different samples (for example, the sample to be analyzed and the control sample) to rule out that the differences in the expression of the biomarkers are due to a different quantity of total protein amount in the sample more than the real differences in the relative levels of expression. The constitutive genes are genes that are always active or being transcribed constantly and which encode for proteins that are expressed constitutively and carry out essential cellular functions. Proteins that are expressed constitutively and can be used in the present invention include, without limitation, β-2-microglobulin (B2M), ubiquitin, 18-S ribosomal protein, cyclophilin, GAPDH, PSMB4, tubulin and actin.

The terms “antibody”, “antibody fragment”, “binds specifically to”, “S100A4 protein”, “diagnosis”, “disease associated to pathogenic fibrosis”, “sample” and “subject” have been defined previously.

All the particular embodiments of the medical uses, diagnostic method and customizing therapy method of the invention are applicable to the use of the kits of the invention.

The invention is described hereinafter by way of the following examples which are to be construed as merely illustrative and not limitative of the invention.

### Materials and Methods

5C3 antibody corresponds to the antibody produced by the hybridoma cell line with deposit number ECACC 10022401 of the European Collection of Cell Cultures (ECACC)

### Example 1: Pro-Fibrotic Effect of S100A4 in Liver

Pathological fibrosis is characterized for crosslinking of extracellular fibers, including collagens and other matrix proteins. This process is finely tuned by several cytokines, growth factors, and accessory cells in a multistep and a multifactor process. Moreover, diagnostic, prognostic, and predictive biomarkers are currently needed to help physicians to early identify pathological fibrosis in such organs.

To define the involved proteins and to identify potential new biomarkers and targets for fibrosis monitoring and treatment, the inventors investigated the effect of the S100A4 protein in the formation of liver fibrosis.

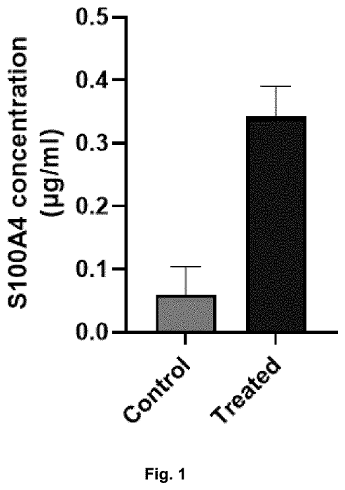

Two different groups of Balb/c mice were administered intraperitoneally with vehicle (PBS), or rhS100A4 (100 μg/animal) in a daily base during 7 days (n=15). At this moment serum concentration of rhS100A4 was analyzed, to prove that animals treated with the S100A4 protein effectively had elevated levels compared to control animals treated with vehicle only. Effectively, as it is shown in FIG. 1, elevated S100A4 serum levels were only present in the group of animals that were administered with rhS100A4, and were practically null in control animals.

Twenty-one days after the start of the study mice were sacrificed and livers were extracted for further analysis. Liver fibrosis was assessed by picrosirius red staining and visualizing the preparations under polarized light, as previously described (Junqueira L C, Bignolas G, Brentani R R. Picrosirius staining plus polarization microscopy, a specific method for collagen detection in tissue sections. Histochem J 1979; 11: 447-455). Picrosirius red staining is one of the best understood histochemical techniques able to selectively highlight collagen networks. It is widely used in the histological visualization of collagen I and Ill fibers in addition to muscle in tissue sections, and it is also a broadly scientific method used in various domains of diagnosis to observe fibrosis levels.

Blinding of the study was ensured during all the experimental setting and in the final analyses.

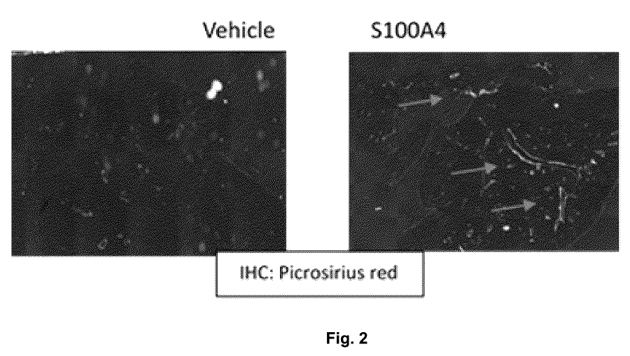

FIG. 2 shows the comparative analysis of the pro-fibrotic activity of S100A4 in the liver of mice. As a consequence of the presence of circulating S100A4, the liver showed an increase of collagen fiber crosslinking that corresponds to an induction of liver fibrosis as it is marked with arrows in the figure.

This experiment demonstrates that an increase in the S100A4 protein circulating levels induces liver fibrosis.

### Example 2: Anti-Liver Fibrosis Effect of 5C3

Pathological fibrosis is currently undertreated since there are few (if any) effective therapeutic options. Fibrotic diseases in the liver, but also in other organs like kidneys, lungs, and heart among others represent a huge unmet medical need.

Once it was demonstrated that S100A4 is directly implicated in the development of fibrosis in the liver of experimental animals, and that it might represent a very useful diagnostic biomarker, the inventors wanted to also investigate whether neutralizing monoclonal antibodies against S100A4 might represent new anti-fibrotic therapeutics.

In this example, mice were divided into three groups (n=15), and were treated intraperitoneally, every day during 7 consecutive days, with:
vehicle (phosphate buffered saline; PBS),
rhS100A4 (100 μg/animal),
a combination of S100A4 (100 μg/animal) and the neutralizing mAb 5C3 (300 μg/animal).

Twenty-one days after the start of the treatment, mice were sacrificed and livers were extracted for its analysis using picrosirius red staining and visualizing the preparations under polarized light to assess liver fibrosis.

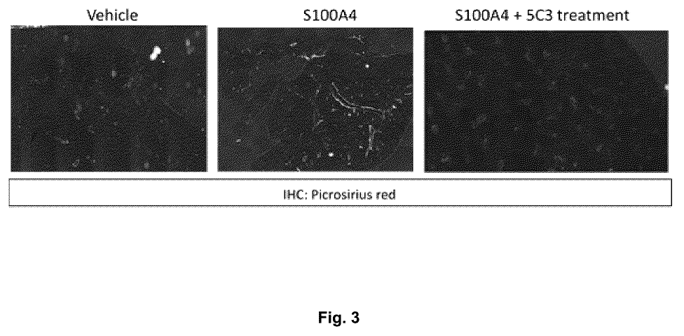

FIG. 3 shows the comparative analysis of the livers from the three groups of animals. It can be clearly stated that S100A4 induces a pro-fibrotic activity in the liver through an increase of collagen fiber crosslinking. Moreover, the efficacy of the anti-S100A4 neutralizing antibody 5C3 to block the extracellular function of S100A4 and eventually liver fibrosis can also be observed in the third group of animals. As can be seen in the figure there is an important reduction in the S100A4-induced liver fibrosis when animals were treated with the combination of the protein with the antibody.

This outcome proves the strong therapeutic potential of the anti-S100A4 strategy to block fibrosis that is dependent of circulating levels of the S100A4 protein. In the study liver fibrosis was analysed as an example, although this therapeutic activity can also be extrapolated to fibrosis in other organs like heart, kidney, lung and many others, provided that it is dependent on increase of circulating or local S100A4 protein levels.

### Example 3: Fibrosis in the Heart is a Consequence of an Increase of S100A4 Levels

To further explore the implication of S100A4 in pathologic fibrosis, and its potential use as a universal therapeutic target to control the disease-related fibrosis, the inventors wanted to determine whether S100A4 is a common mechanism and/or a triggering molecule of fibrosis in other organs.

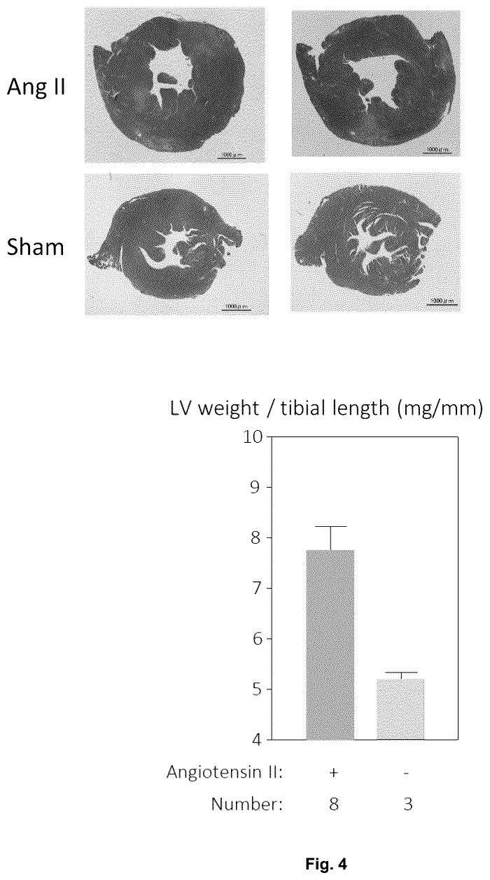

First, the chronic angiotensin II-infusion model in mice as a well-known and established model of cardiac fibrosis was investigated. In this model, as it can be observed in FIG. 4, the infusion of angiotensin II (0.96 mg/kg/day) for 2 weeks via subcutaneously implanted mini-osmotic pumps in mice anesthetized with intraperitoneal injection of ketamine and xylazine, leads to a left ventricle (LV) hypertrophy and cardiac fibrosis, compared to vehicle (sham) treated animals. This might be quantified through the measurement of LV weight (mg) and normalized to the tibial length (mm).

Moreover, in the same model, both S100A4 as well as RAGE (one of the known receptors of S100A4) were also measured through quantitative real-time RT-PCR. Total RNA was isolated using TRIzol reagent and treated with DNase I. cDNA was generated using SuperScript Ill reverse transcriptase. Specific genes were amplified using PCR Master Mix. Expression of both genes was normalized to GAPDH. This protocol was also followed for the measurements of other related molecules described in the following figures.

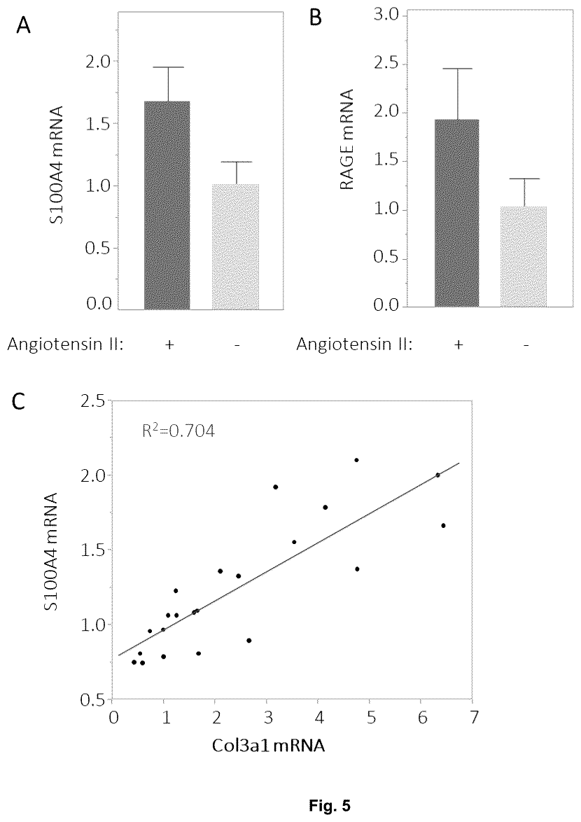

The outcome of the analysis of S100A4 and RAGE can be observed in FIG. 5, where clearly a marked increase of these molecules coincides with the fibrosis induction with Angiotensin II treatment. The correlation with fibrosis is further demonstrated in the FIG. 5C where S100A4 measurements were plotted in front of Col3a1 measurements, a known biomarker of fibrosis.

### Example 4: Anti-Cardiac Fibrosis Effect of 5C3

Once it was demonstrated that S100A4 is directly implicated in the development of fibrosis in the heart of experimental animals, and that it might represent a very useful diagnostic biomarker, the inventors wanted to also investigate whether neutralizing monoclonal antibodies anti-S100A4 might represent new anti-fibrotic therapeutics.

In this example, mice were divided into four groups following the next scheme:
Angiotensin II minipump infusion+vehicle treatment
Angiotensin II minipump infusion+anti-S100A4 mAb 5C3 treatment
Vehicle minipump infusion+vehicle treatment
Vehicle minipump infusion+anti-S100A4 mAb 5C3 treatment

Treatments consisted of intraperitoneal administrations of vehicle (PBS) or mAb 5C3 (at 20 mg/Kg) three days per week.

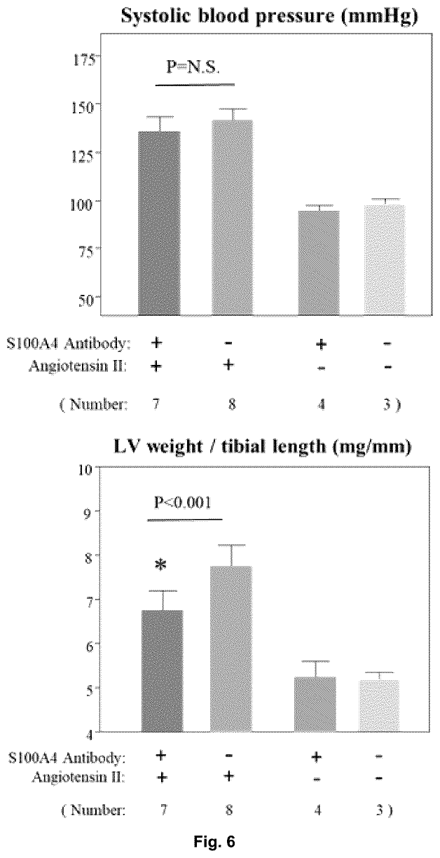

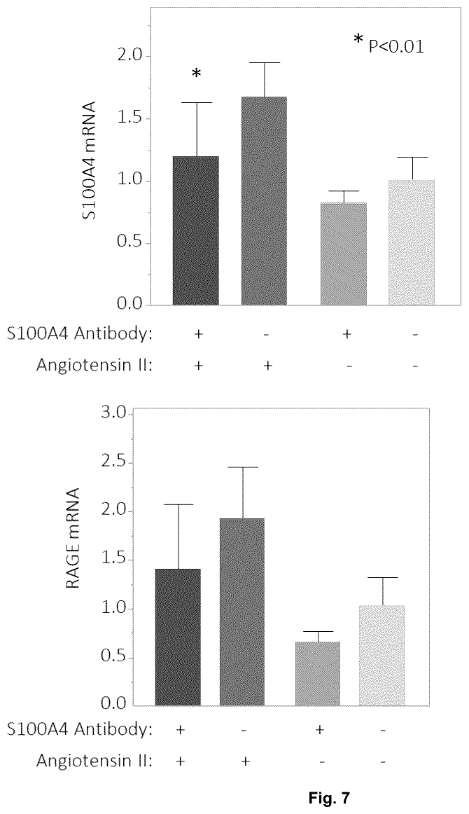

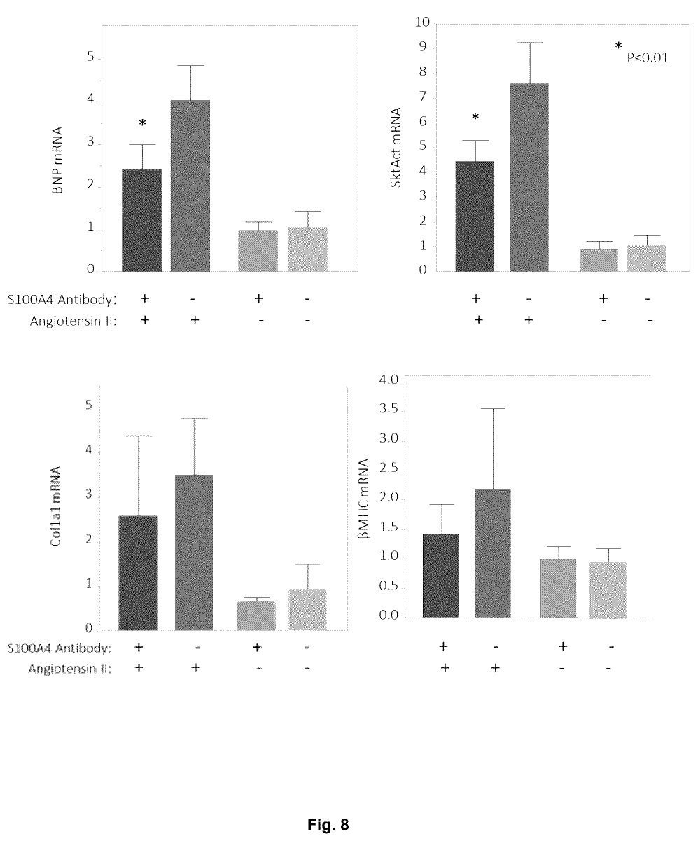

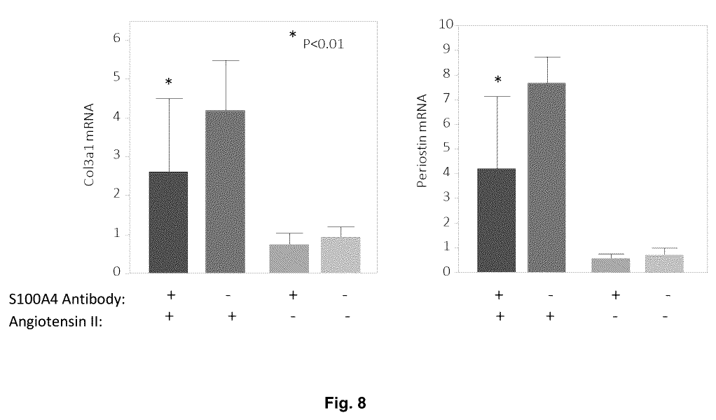

To analyze the anti-fibrotic effect of the antibody, several fibrosis related molecules as well as S100A4 and RAGE were measured through qPCR at the end of the experimental setting. The outcomes of these analysis, showed in FIG. 6, FIG. 7, and FIG. 8, clearly demonstrated that:
Levels of S100A4 and RAGE raise in cardiac fibrosis
mAb 5C3 treatment significantly inhibit the increase of fibrotic biomarkers as well as cardiac fibrosis (LV weight/tibial length ratio)
mAb 5C3 treatment did not affect physiological parameters like systolic blood pressure

These outcomes prove the strong therapeutic potential of the anti-S100A4 strategy to block fibrosis that is dependent of circulating levels of the S100A4 protein. In this study the inventors analysed cardiac fibrosis as a second example, although this therapeutic activity can also be extrapolated to fibrosis in other organs like liver, kidney, lung, and many others, provided that it is dependent on increase of circulating or local S100A4 levels.

1: A method for the prevention and/or treatment of a disease associated to pathogenic fibrosis comprising: administering an antibody or a fragment thereof that binds specifically to the S100A4 protein to a subject in need thereof.

2: The method according to claim 1, wherein the pathogenic fibrosis is associated to increased levels of S100A4.

3: The method according to claim 1, wherein the disease is not silicon-induced fibrosis, asbestos-induced fibrosis or cystic fibrosis.

4: The method according to claim 1, wherein the disease is not associated to metastasis.

5: The method according to claim 1, wherein the disease is selected from the group consisting of liver fibrosis, cardiac fibrosis, kidney fibrosis and lung fibrosis.

6: The method according to claim 5, wherein the disease is selected from the group consisting of liver fibrosis and cardiac fibrosis.

7: The method according to claim 6, wherein the disease is cardiac fibrosis.

8: The method according to claim 1, wherein the antibody is selected from the group consisting of: an antibody that recognizes an epitope of S100A4 comprising the sequence ELPSFLGKRT (SEQ ID NO: 1), an antibody that recognizes an epitope of S100A4 comprising the sequence EGFPDKQPRKK (SEQ ID NO: 2) and the antibody produced by the hybridoma ECACC 11051804; and/or wherein the CDRs of the light chain of the variable region of the antibody against S100A4 comprise the sequences set forth in SEQ ID NO: 3, 4 and 5, or a functionally equivalent variant thereof, and the CDRs of the heavy chain of the variable region comprise the sequences set forth in SEQ ID NO: 6, 7, 8 or a functionally equivalent variant thereof; and/or the FR region of the light chain of the variable region of the antibody against S100A4 comprises the sequences set forth in SEQ ID NO: 9, 10, 11 and 12 or a functionally equivalent variant thereof, and the FR region of the heavy chain of the variable region of the antibody against S100A4 comprises the sequences set forth in SEQ ID NO: 13, 14, 15 and 16 or a functionally equivalent variant thereof, and/or the antibody against S100A4 comprises at least a VL region comprising the sequence set forth in SEQ ID NO: 17 or a functionally equivalent variant thereof and at least a VH region comprising the sequence set forth in SEQ ID NO: 18 or a functionally equivalent variant thereof: or wherein the antibody against S100A4 is a monoclonal antibody produced by the hybridoma cell line deposited with the accession number ECACC 10022401.

9-12. (canceled)

13: The method according to claim 1, wherein the antibody against S100A4 is a monoclonal antibody produced by the hybridoma cell line deposited with the accession number ECACC 11051801, ECACC 11051802, ECACC 11051803, or ECACC 11051804.

14: An in vitro method for diagnosing a disease associated to pathogenic fibrosis in a subject which comprises:
a) Detecting the levels of the S100A4 protein or of a variant thereof in a sample of said subject, and
b) Comparing said levels with a reference value,
wherein increased levels of the S100A4 protein or of a variant thereof with respect to the reference value are indicative of the subject suffering from a disease associated to pathogenic fibrosis.

15: The in vitro method for diagnosing a disease associated to pathogenic fibrosis according to claim 14, wherein the fibrosis is associated to increased levels of S100A4.

16: The in vitro method for diagnosing a disease associated to pathogenic fibrosis according to claim 14, wherein the disease is not silicon-induced fibrosis, asbestos-induced fibrosis or cystic fibrosis.

17: The in vitro method for diagnosing a disease associated to pathogenic fibrosis according to claim 14, wherein the disease is not associated to metastasis.

18: The in vitro method for diagnosing a disease associated to pathogenic fibrosis according to claim 14, wherein the disease is selected from the group consisting of liver fibrosis, cardiac fibrosis, kidney fibrosis and lung fibrosis.

19: The in vitro method for diagnosing a disease associated to pathogenic fibrosis according to claim 18, wherein the disease is selected from the group consisting of cardiac fibrosis and liver fibrosis.

20: The in vitro method for diagnosing a disease associated to pathogenic fibrosis according to claim 19, wherein the disease is cardiac fibrosis.

21: The in vitro method for diagnosing a disease associated to pathogenic fibrosis according to claim 14 wherein the levels of the S100A4 protein or of a variant thereof are detected by means of using an antibody or a fragment thereof that binds specifically to the S100A4 protein.

22: The in vitro method for diagnosing a disease associated to pathogenic fibrosis according to claim 21 wherein the antibody recognizes an epitope of S100A4 comprising the sequence ELPSFLGKRT (SEQ ID NO: 1), an epitope of S100A4 comprising the sequence EGFPDKQPRKK (SEQ ID NO: 2) or is the antibody produced by the hybridoma ECACC 11051804; and/or wherein the CDRs of the light chain of the variable region of the antibody against S100A4 comprise the sequences set forth in SEQ ID NO: 3, 4 and 5 or a functionally equivalent variant thereof, and the CDRs of the heavy chain of the variable region comprise the sequences set forth in SEQ ID NO: 6, 7, 8 or a functionally equivalent variant thereof; and/or wherein the FR region of the light chain of the variable region of the antibody against S100A4 comprises the sequences set forth in SEQ ID NO: 9, 10, 11 and 12 or a functionally equivalent variant thereof, and the FR region of the heavy chain of the variable region of the antibody against S100A4 comprises the sequences set forth in SEQ ID NO: 13, 14, 15 and 16 or a functionally equivalent variant thereof, and/or the antibody against S100A4 comprises at least a VL region comprising the sequence set forth in SEQ ID NO: 17 or a functionally equivalent variant thereof and at least a VH region comprising the sequence set forth in SEQ ID NO: 18 or a functionally equivalent variant thereof: or wherein the antibody against S100A4 is a monoclonal antibody produced by the hybridoma cell line deposited with the accession number ECACC 10022401, ECACC 11051801, ECACC 11051802, ECACC 11051803 or ECACC 11051804.

23-27. (canceled)

28: The in vitro method for diagnosing a disease associated to pathogenic fibrosis according to claim 21 wherein the sample is serum or plasma.

29: An in vitro method for designing a customized therapy for a subject diagnosed with a disease associated to pathogenic fibrosis which comprises
a) detecting the levels of the S100A4 protein or of a variant thereof in a sample of said subject, and
b) comparing said levels with a reference value,
wherein increased levels of the S100A4 protein or of a variant thereof with respect to the reference value are indicative that the subject is to be treated with an antibody or a fragment thereof that binds specifically to the S100A4 protein.

30: (canceled)
